(19) United States
(12) Patent Application Publication
(10) Pub. No.: US 2023/0406949 A1
(43) Pub. Date: Dec. 21, 2023

(54) COMBINATORIAL IMMUNOTHERAPEUTIC METHODS AND COMPOSITIONS FOR PANCREATIC DUCTAL ADENOCARCINOMA TREATMENT

(71) Applicant: BOARD OF REGENTS, THE UNIVERSITY OF TEXAS SYSTEM, Austin, TX (US)

(72) Inventors: Ronald DEPINHO, Houston, TX (US); Pat GULHATI, Houston, TX (US)

(73) Assignee: BOARD OF REGENTS, THE UNIVERSITY OF TEXAS SYSTEM, Austin, TX (US)

(21) Appl. No.: 18/248,778

(22) PCT Filed: Oct. 12, 2021
(86) PCT No.: PCT/US2021/054577

§ 371 Date: Apr. 12, 2023

Related U.S. Application Data
(60) Provisional application No. 63/091,065, filed on Oct. 13, 2020.

(51) Int. Cl. C07K 16/28; A61K 31/537; A61K 31/513; A61K 31/519; A61K 45/06; A61K 31/4745; A61K 31/555; A61K 31/337; A61K 31/7068

(52) U.S. Cl. CPC C07K 16/2878; C07K 16/2803; A61K 31/537; A61K 31/513; A61K 31/519; A61K 45/06; A61K 31/4745; A61K 31/555; A61K 31/337; A61K 31/7068; A61K 2039/507

(57) ABSTRACT

Aspects of the present disclosure are directed to immunotherapeutic methods for treating a subject having PDAC. Disclosed are methods comprising treatment with two or more immunotherapeutic agents for generating an effective immune response to PDAC. Certain aspects relate to methods comprising the use of a LAG-3 antagonist and a 41BB agonist, in some cases together with a chemokine receptor inhibitor, for the treatment of PDAC. Also disclosed are compositions comprising a LAG-3 antagonist and a 41BB agonist. In some cases the disclosed compositions further comprise a chemokine receptor inhibitor.

### CROSS REFERENCE TO RELATED APPLICATIONS

This application claims benefit of priority to U.S. Provisional Application No. 63/091,065, filed Oct. 13, 2020, which is hereby incorporated by reference in its entirety.

This invention was made with government support under CA117969 awarded by the National Cancer Institute. The government has certain rights in the invention.

### BACKGROUND

### I. Field of the Invention

Aspects of this invention relate to at least the fields of cancer biology, immunology, and medicine.

### II. Background

Pancreatic ductal adenocarcinoma (PDAC) is among the most lethal human cancers, with a 5-year overall survival (OS) rate of 9% [1]. The mainstay of treatment for metastatic PDAC is chemotherapy with gemcitabine- or fluorouracil-based regimens; however, chemotherapy benefit is often modest and transient [2, 3]. Immune checkpoint therapy (ICT) has transformed treatment and survival for a number of advanced cancers; however, PDAC has been considered ‘non-immunogenic’ as multiple trials have shown that PDAC is recalcitrant to currently available ICT, including anti-PD-L1 and anti-CTLA4 based therapies [4, 5, 6, 7, 8]. There is a need in the art for novel therapeutic strategies that can transform PDAC from “non-immunogenic” to immunogenic, thereby providing effective immunotherapeutic treatment and significantly improving outcomes.

### SUMMARY

The present disclosure fulfils certain unmet needs in the art by providing immunotherapeutic agents and methods for treatment of PDAC. Aspects of the present disclosure are directed to immunotherapeutic methods for the treatment of PDAC. Further aspects are directed to immunotherapeutic compositions. As disclosed herein, such methods and compositions unexpectedly render PDAC treatable, durably prolong survival, and are curative in some embodiments. Methods of the disclosure, in some embodiments, provide significantly improved outcomes for PDAC patients compared with currently available treatments. Certain aspects pertain to combination treatment strategies comprising the use of a LAG-3 antagonist, a 41BB agonist, and a chemokine receptor (e.g., CCR2) inhibitor for treatment of PDAC.

Embodiments of the disclosure include methods for treating a subject for PDAC, methods for stimulating an immune response to PDAC, methods for cancer immunotherapy, methods for stimulating immune cell activation, methods for inhibiting myeloid immunosuppressive cells, immunotherapeutic compositions, and pharmaceutical compositions. Methods of the disclosure can include 1, 2, 3 or more of the following steps: administering a LAG-3 antagonist, administering a 41BB agonist, administering a chemokine receptor inhibitor, administering a CCR2 inhibitor, administering an arginase inhibitor, administering a chemotherapy, administering a radiotherapy, administering an immunotherapy, diagnosing a subject with PDAC, and identifying a subject as being a candidate for a combination therapy. Compositions of the disclosure may comprise 1, 2, 3, or more of the following components: a LAG-3 antagonist, an anti-LAG-3 antibody, a 41BB agonist, an anti-41BB antibody, a chemokine receptor inhibitor, a CCR1 inhibitor, a CCR2 inhibitor, a Cxcr2 inhibitor, an arginase inhibitor, an Arg1 inhibitor, and a pharmaceutically acceptable excipient. Any one or more of the preceding steps or components may be excluded from certain embodiments of the disclosure.

Disclosed herein, in some embodiments, is a method for treating a subject for PDAC, the method comprising administering to the subject (a) a 41BB agonist; (b) a LAG3 antagonist; and (c) a chemokine receptor inhibitor. In some embodiments, the method further comprises administering to the subject an additional chemokine receptor inhibitor. In some embodiments, the method further comprises administering to the subject an arginase inhibitor. In some embodiments, the arginase inhibitor is an Arg1 inhibitor. In some embodiments, the method further comprises administering to the subject a Cxcr2 inhibitor. In some embodiments, the Cxcr2 inhibitor is an anti-Cxcr2 antibody. In some embodiments, the anti-Cxcr2 antibody is MAB2164. In some embodiments, the method comprises inhibiting growth, proliferation, and/or immunosuppressive activity of myeloid cells in the subject. In some embodiments, the method further comprises administering to the subject an additional cancer therapy. In some embodiments, the additional cancer therapy comprises chemotherapy, radiotherapy, or immunotherapy. In some embodiments, the additional cancer therapy is FOLFIRINOX. In some embodiments, the additional cancer therapy is gemcitabine. In some embodiments, the additional cancer therapy is gemcitabine with nab-paclitaxel. In some embodiments, the gemcitabine is administered to the subject prior to administering the 41BB agonist, the LAG3 antagonist, and the chemokine receptor inhibitor. In some embodiments, the method does not comprise administering to the subject any additional cancer therapy. In some embodiments, the subject was previously treated for PDAC with a previous treatment. In some embodiments, the subject was determined to be resistant to the previous treatment. In some embodiments, the previous treatment comprised FOLFIRINOX. In some embodiments, the previous treatment comprised gemcitabine. In some embodiments, the previous treatment comprised gemcitabine with nab-paclitaxel. In some embodiments, the previous treatment comprised a PD-1 antagonist, a PD-L1 antagonist, or a CTLA-4 antagonist. In some embodiments, the 41BB agonist, the LAG3 antagonist, and the chemokine receptor inhibitor are administered substantially simultaneously. In some embodiments, the 41BB agonist, the LAG3 antagonist, and the chemokine receptor inhibitor are administered sequentially. In some embodiments, the 41BB agonist, the LAG3 antagonist, and the chemokine receptor inhibitor are administered in a single composition. In some embodiments, the 41BB agonist, the LAG3 antagonist, and the chemokine receptor inhibitor are administered in two or more different compositions.

Disclosed herein, in some embodiments, is a composition comprising (a) an anti-41BB agonist, (b) an anti-LAG3 antagonist, and (c) a chemokine receptor inhibitor. In some embodiments, the composition further comprises an arginase inhibitor. In some embodiments, the arginase inhibitor is an Arg1 inhibitor. In some embodiments, the composition further comprises an iNOS inhibitor. In some embodiments, the composition further comprises a Cxcr2 inhibitor. In some embodiments, the Cxcr2 inhibitor is an anti-Cxcr2 antibody. In some embodiments, the anti-Cxcr2 antibody is MAB2164. In some embodiments, the composition further comprises a pharmaceutically acceptable excipient.

In some embodiments, the 41BB agonist is an anti-41BB antibody. In some embodiments, the 41BB antibody is LOB12.3. In some embodiments, the LAG3 antagonist is an anti-LAG3 antibody. In some embodiments, the LAG3 antagonist is C9B7W. In some embodiments, the chemokine receptor inhibitor is a CCR1 inhibitor. In some embodiments, the chemokine receptor inhibitor is a CCR2 inhibitor. In some embodiments, the chemokine receptor inhibitor is RS504393.

Embodiments of the disclosure are directed to a method for treating a subject for pancreatic ductal adenocarcinoma, the method comprising administering to the subject (a) a 41BB agonist; (b) a LAG3 antagonist; (c) a CCR2 inhibitor.

Further embodiments are directed to a pharmaceutical composition comprising (a) a 41BB agonist; (b) a LAG3 antagonist; and (c) a CCR2 inhibitor; and (d) a pharmaceutically acceptable excipient.

“Individual, “subject,” and “patient” are used interchangeably and can refer to a human or non-human.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

A variety of embodiments are discussed throughout this application. Any embodiment discussed with respect to one aspect applies to other aspects as well and vice versa. Each embodiment described herein is understood to be embodiments that are applicable to all aspects. It is contemplated that any embodiment discussed herein can be implemented with respect to any method or composition, and vice versa. Furthermore, compositions and kits can be used to achieve methods disclosed herein.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1I show results from characterization of the iKRAS PDAC mouse model.

FIGS. 2A-2F show results from CyTOF profiling of cells from the tumor microenvironment of iKRAS PDAC tumors.

FIGS. 3A-3F show results from analysis of data from single cell RNA sequencing of immune cells from iKRAS PDAC tumors.

FIGS. 4A-4F show results from analysis of data from single cell RNA sequencing of immune cells from iKRAS PDAC tumors.

FIGS. 5A-5F show results from analysis of data from single cell RNA sequencing of immune cells from iKRAS PDAC mice treated with various ICT regimens.

FIGS. 6A-6H show results from analysis of iKRAS PDAC mice treated with various ICT regimens and results from multiplex immunohistochemistry staining of human PDAC tissue specimens.

FIGS. 7A-7C show results from analysis of sequencing data from human PDAC cohorts from TCGA and ICGC datasets. FIGS. 7D-7F show results from analysis of the immunosuppressive activity of intratumoral CD11b⁺Gr1⁺ cells from iKRAS tumors. FIG. 7G shows a schematic of the anti-Gr1 antibody treatment regimen. FIGS. 7H-7I show results from analysis of data from single cell RNA sequencing of myeloid immune cells from iKRAS PDAC tumors. FIG. 7J shows a schematic of the combination treatment regimen (anti-LAG-3, anti-41BB, CCR2 inhibitor).

FIGS. 8A-8B show results from analysis of anti-Gr1-treated iKRAS PDAC mice. FIGS. 8C-8D show results from clustering analysis of intrinsic myeloid cell heterogeneity in iKRAS PDAC tumors. FIGS. 8E-8F show results from treatment of iKRAS PDAC mice with a CCR2 inhibitor, RS504393, in combination with agonist 41BB and antagonist LAG3 antibodies.

### DETAILED DESCRIPTION

Aspects of the present disclosure address needs in the art by providing methods and compositions for treatment of PDAC. The present disclosure is based, at least in part, on the surprising and unexpected discovery that providing a novel combination of a 41BB agonist, a LAG3 antagonist, and a chemokine receptor (e.g., CCR1, CCR2) inhibitor provides significant and synergistic efficacy in treating a subject for previously untreatable PDAC. Aspects of the disclosure are directed to methods for treating a subject for PDAC comprising administering an effective amount of a 41BB agonist, a LAG3 antagonist, and a chemokine receptor inhibitor. Also described are compositions comprising a 41BB agonist, a LAG3 antagonist, and a chemokine receptor inhibitor.

### I. Therapeutic Methods

The compositions of the disclosure may be used for in vivo, in vitro, or ex vivo administration. The route of administration of the composition may be, for example, intracutaneous, subcutaneous, intravenous, local, topical, oral, and intraperitoneal administrations.

A. Cancer Therapy

In some embodiments, the disclosed method comprise administering a cancer therapy to a subject. The cancer therapy may be chosen based on, for example, expression level measurements (e.g., biomarker expression levels), alone or in combination with a clinical risk score calculated for the subject. In some embodiments, the cancer therapy comprises a local cancer therapy. In some embodiments, the cancer therapy comprises a systemic cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be an immune checkpoint therapy. In some embodiments, the cancer therapy comprises use of a chemokine receptor inhibitor. A chemokine receptor inhibitor may be an inhibitor of a chemokine receptor such as, for example, CCR1, CCR2, CCR3, CCR4, CCR5, CCR6, CCR7, CCR8, CCR9, CCR10, Cxcr1, Cxcr2, Cxcr3, Cxcr4, Cxcr5, or Cxcr6. In some embodiments, the cancer therapy comprises an inhibitor of growth, proliferation, and/or immunosuppressive activity of myeloid cells in the subject. In some embodiments, the cancer therapy comprises an arginase (e.g., Arg1) inhibitor. In some embodiments, the cancer therapy comprises an inducible nitric oxide synthase (iNOS) inhibitor. In some embodiments, the cancer therapy comprises a Cxcr2 inhibitor (e.g., an anti-Cxcr2 antibody). Any of these cancer therapies may also be excluded. Combinations of these therapies may also be administered.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma; ovarian stromal tumor; thecoma; granulosa cell tumor; androblastoma; sertoli cell carcinoma; leydig cell tumor; lipid cell tumor; paraganglioma; extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; blue nevus; sarcoma; fibrosarcoma; fibrous histiocytoma; myxosarcoma; liposarcoma; leiomyosarcoma; rhabdomyosarcoma; embryonal rhabdomyosarcoma; alveolar rhabdomyosarcoma; stromal sarcoma; mixed tumor; mullerian mixed tumor; nephroblastoma; hepatoblastoma; carcinosarcoma; mesenchymoma; brenner tumor; phyllodes tumor; synovial sarcoma; mesothelioma; dysgerminoma; embryonal carcinoma; teratoma; struma ovarii; choriocarcinoma; mesonephroma; hemangiosarcoma; hemangioendothelioma; kaposi's sarcoma; hemangiopericytoma; lymphangiosarcoma; osteosarcoma; juxtacortical osteosarcoma; chondrosarcoma; chondroblastoma; mesenchymal chondrosarcoma; giant cell tumor of bone; ewing's sarcoma; odontogenic tumor; ameloblastic odontosarcoma; ameloblastoma; ameloblastic fibrosarcoma; pinealoma; chordoma; glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma; neurofibrosarcoma; neurilemmoma; granular cell tumor; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, disclosed are methods for treating cancer originating from the pancreas. In some embodiments, the cancer is pancreatic cancer. In some embodiments, the cancer is pancreatic ductal adenocarcinoma (PDAC).

B. Cancer Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and examples are described below.

1. Checkpoint Inhibitors and Combination Treatment

Aspects of the disclosure may include administration of immune checkpoint inhibitors, examples of which are further described below. As disclosed herein, “checkpoint inhibitor therapy” (also “immune checkpoint blockade therapy,” “checkpoint blockade therapy,” “immune checkpoint therapy,” “ICT,” “checkpoint blockade immunotherapy,” or “CBI”), refers to cancer therapy comprising providing one or more immune checkpoint inhibitors to a subject suffering from or suspected of having cancer.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

c. LAG-3

Another immune checkpoint that can be targeted in the methods provided herein is lymphocyte-activation gene 3 protein (LAG-3; also LAG3), also known as CD223. The complete cDNA sequence of human LAG-3 has the Genbank accession number NC_000012. LAG-3 is an inhibitory receptor on antigen-activated T-cells. Following TCR engagement, LAG3 associates with CD3-TCR in the immunological synapse and directly inhibits T-cell activation. Targeting molecules of the disclosure may block one or more functions of LAG-3. In some embodiments, the present disclosure provides immune checkpoint inhibitors, wherein the immune checkpoint inhibitor is a LAG-3 antagonist. As used herein, a “LAG3 antagonist” describes any molecule capable of reducing or preventing LAG-3 signaling activity in a cell. For example, a LAG-3 antagonist may be a LAG-3 antibody capable of blocking an interaction between LAG-3 and MHC class II.

In some embodiments, the immune checkpoint inhibitor is an anti-LAG-3 antibody (e.g., a human antibody, a humanized antibody, or a chimeric antibody), an antigen binding fragment thereof, an immunoadhesin, a fusion protein, or oligopeptide.

Anti-human-LAG-3 antibodies (or VH and/or VL domains derived therefrom) suitable for use in the present methods can be generated using methods well known in the art. Alternatively, art recognized anti-LAG-3 antibodies can be used. For example, an anti-LAG-3 antibody useful as a checkpoint inhibitor in the methods and compositions of the disclosure is relatlimab (also known as BMS-986016). In another example, an anti-LAG-3 antibody useful in the methods and compositions of the disclosure is C9B7W (also “clone C9B7W”, described in, for example, Workman C J, et al. Eur J Immunol. 2002; 32(8):2255-2263, incorporated by reference herein in its entirety).

In some embodiments, the LAG3 antagonist comprises the heavy and light chain CDRs or VRs of relatlimab. Accordingly, in one embodiment, the LAG3 antagonist comprises the CDR1, CDR2, and CDR3 domains of the VH region of relatlimab, and the CDR1, CDR2 and CDR3 domains of the VL region of relatlimab. In some embodiments, the LAG3 antagonist comprises the heavy and light chain CDRs or VRs of C9B7W. Accordingly, in one embodiment, the LAG3 antagonist comprises the CDR1, CDR2, and CDR3 domains of the VH region of relatlimab, and the CDR1, CDR2 and CDR3 domains of the VL region of C9B7W. In another embodiment, the antibody competes for binding with and/or binds to the same epitope on LAG-3 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+ Th1 and CD8+ Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+ Th1-specific cell surface protein that regulates macrophage activation. Inhibitors of the disclosure may block one or more functions of TIM-3 activity.

In some aspects, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or V_{H} and/or V_{L} domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to TIM-3 also can be used.

In some aspects, the inhibitor comprises the heavy and light chain CDRs or VRs of an anti-TIM-3 antibody. Accordingly, in one aspect, the inhibitor comprises the CDR1, CDR2, and CDR3 domains of the V_{H} region of an anti-TIM-3 antibody, and the CDR1, CDR2 and CDR3 domains of the V_{L} region of an anti-TIM-3 antibody. In another aspect, the antibody has at least about 70, 75, 80, 85, 90, 95, 97, or 99% (or any derivable range or value therein) variable region amino acid sequence identity with the above-mentioned antibodies.

2. Activation of Co-Stimulatory Molecules

In some embodiments, the immunotherapy comprises an activator (also “agonist”) of a co-stimulatory molecule. In some embodiments, the activator comprises an activator of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Activators include stimulatory antibodies, polypeptides, compounds, and nucleic acids.

In some embodiments, disclosed are immunotherapeutic methods comprising a 4-1BB (also “41BB”) agonist. 41BB is also known as CD137 or TNFRSF9. The complete cDNA sequence of human 41BB has the Genbank accession number NM_001561. As used herein, a “41BB agonist” describes any molecule capable of stimulating or enhancing 41BB signaling activity in a cell. For example, a 41BB agonist may be a 41BB antibody capable of activating 41BB signaling. In another example a 41BB agonist is 41BB ligand (41BBL).

Anti-human-41BB antibodies (or VH and/or VL domains derived therefrom) suitable for use in the present methods can be generated using methods well known in the art. Alternatively, art recognized anti-41BB antibodies can be used. For example, an anti-41BB antibody useful in the methods and compositions of the disclosure is utomilumab (also known as PF-05082566). In another example, an anti-41BB antibody useful in the methods and compositions of the disclosure is urelumab. In another example, an anti-41BB antibody useful in the methods and compositions of the disclosure is LOB12.3 (also “clone LOB12.3”, described in, for example, Taraban, Vadim Y et al. Eur J Immunol. 2002; 32(12):3617-3627, incorporated by reference here in it its entirety).

In some embodiments, the 41BB agonist comprises the heavy and light chain CDRs or VRs of utomilumab or urelumab. Accordingly, in one embodiment, the 41BB agonist comprises the CDR1, CDR2, and CDR3 domains of the VH region of utomilumab, urelumab, or LOB12.3, and the CDR1, CDR2 and CDR3 domains of the VL region of utomilumab, urelumab, or LOB12.3. In another embodiment, the antibody competes for binding with and/or binds to the same epitope on 41BB as the above-mentioned antibodies. In another embodiment, the antibody has at least about 70, 75, 80, 85, 90, 95, 97, or 99% (or any derivable range therein) variable region amino acid sequence identity with the above-mentioned antibodies.

3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell, natural killer (NK) cell, or other immune cell. The receptors are called chimeric because they are fused of parts from different sources. CAR-T cell therapy refers to a treatment that uses such transformed cells for cancer therapy, where the transformed cells are T cells. Similar therapies include, for example, CAR-NK cell therapy, which uses transformed NK cells.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

C. Chemokine Receptors and Inhibitors

Embodiments of the disclosure may include administration of inhibitors of one or more chemokine receptors. A “chemokine receptor inhibitor” describes any agent or molecule capable of inhibiting the activity of one or more chemokine receptors.

Chemokine receptors are transmembrane, G protein-coupled receptors activated by binding of one or more chemokines. Chemokine receptors are expressed by a variety of immune cells, including myeloid cells such as macrophages. Chemokine receptors include, for example, CCR1, CCR2, CCR3, CCR4, CCR5, CCR6, CCR7, CCR8, CCR9, CCR10, Cxcr1, Cxcr2, Cxcr3, Cxcr4, Cxcr5, and Cxcr6. In some embodiments, a chemokine receptor inhibitor of the present disclosure is a CCR1 and/or CCR2 inhibitor. In some embodiments, a chemokine receptor inhibitor of the present disclosure is a Cxcr2 inhibitor.

In some embodiments, a chemokine receptor inhibitor of the present disclosure is a CCR2 inhibitor. C—C chemokine receptor 2 (CCR2) signaling plays a key role in the recruitment of myeloid cells to tumors [21, 22]. Aspects of the present disclosure are directed to methods for PDAC treatment comprising administration of a CCR2 inhibitor. In some embodiments, a CCR2 inhibitor is an anti-CCR2 antibody or antigen binding fragment thereof. In some embodiments, a CCR2 inhibitor is a small molecule CCR2 inhibitor. One example of a CCR2 inhibitor is RS504393. Various CCR2 inhibitors are known in the art and are contemplated herein.

In some embodiments, a chemokine receptor inhibitor of the present disclosure is a Cxcr2 inhibitor. C—X—C chemokine receptor 2 (Cxcr2) is a receptor for interleukin 8. Aspects of the present disclosure are directed to methods for pancreatic cancer treatment comprising administration of a Cxcr2 inhibitor. In some embodiments, a Cxcr2 inhibitor is an anti-Cxcr2 antibody or antigen binding fragment thereof. One example of an anti-Cxcr2 antibody is MAB2164.

D. Chemotherapies

Aspects of the disclosure comprise chemotherapies and methods for use. In some embodiments, methods of the disclosure comprise administrating a chemotherapy to a subject. In some embodiments, a chemotherapy is administered to a subject in combination with one or more therapeutics disclosed herein (e.g., a 41BB agonist, a LAG3 antagonist, a chemokine receptor inhibitor, etc.). Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, gemcitibine is a particularly suitable chemotherapeutic agent, with or without albumin-bound paclitaxel (also “nab-paclitaxel”).

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in certain embodiments of the present disclosure, with or without nab-paclitaxel.

In some embodiments, multiple chemotherapies are provided to a patient as a chemotherapeutic regimen. In some embodiments, a chemotherapy is a combination of folinic acid, 5-fluorouracil (5-FU), irinotecan, and oxaliplatin. Such a treatment regimen may be referred to as “FOLFIRINOX”.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### II. Administration of Therapeutic Compositions

The therapy provided herein may comprise administration of a combination of therapeutic agents, such as a first therapeutic agent (e.g., a 41BB agonist), a second therapeutic agent (e.g., a LAG-3 antagonist), and/or a third therapeutic agent (e.g., a chemokine receptor inhibitor such as a CCR2 inhibitor). The therapies may be administered in any suitable manner known in the art. For example, the first, second, and third therapeutic agents may be administered sequentially (at different times) or concurrently (at the same time). In some embodiments, the first, second, and third therapeutic agents are administered in a separate composition. In some embodiments, the first, second, and third therapeutic agents are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### III. Kits

Certain aspects of the present invention also concern kits containing compositions of the invention or compositions to implement methods of the invention. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit embodiments.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—CyTOF Analysis of iKRAS PDAC Tumor Microenvironment

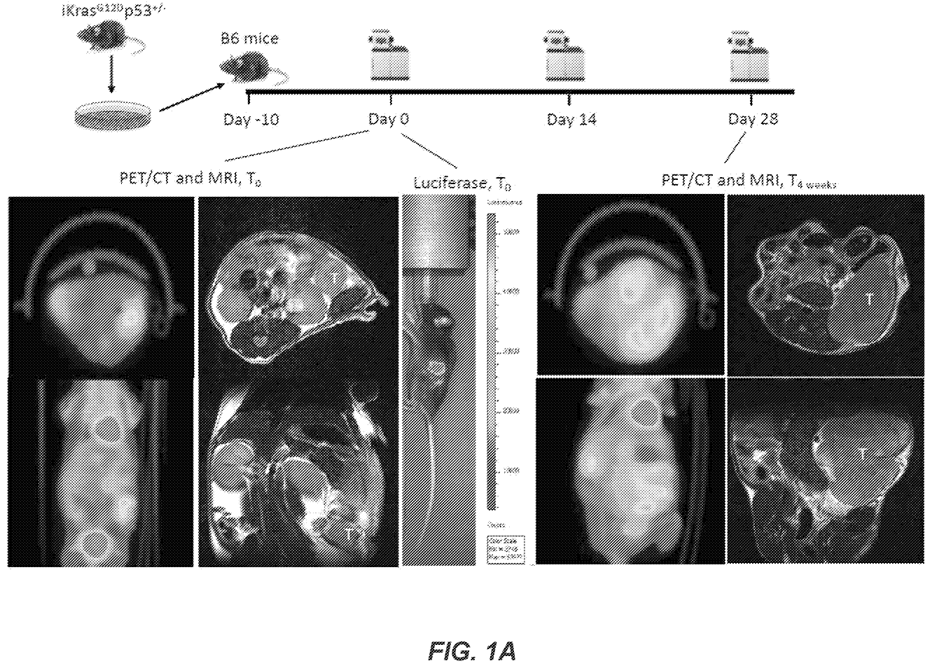

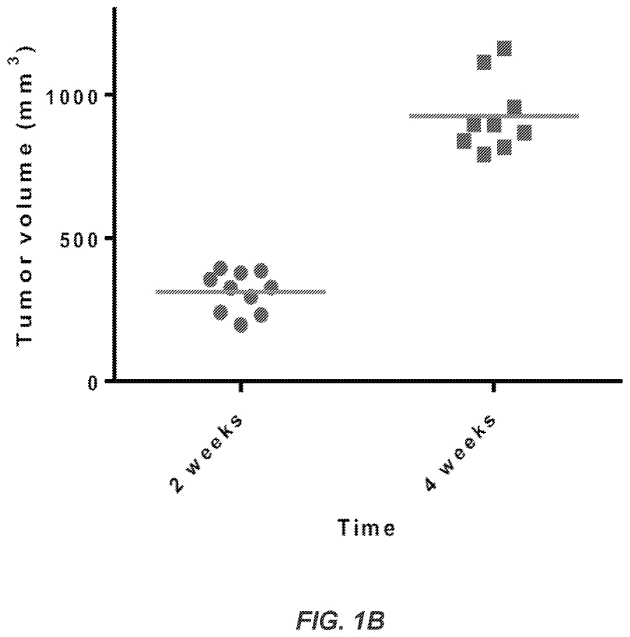

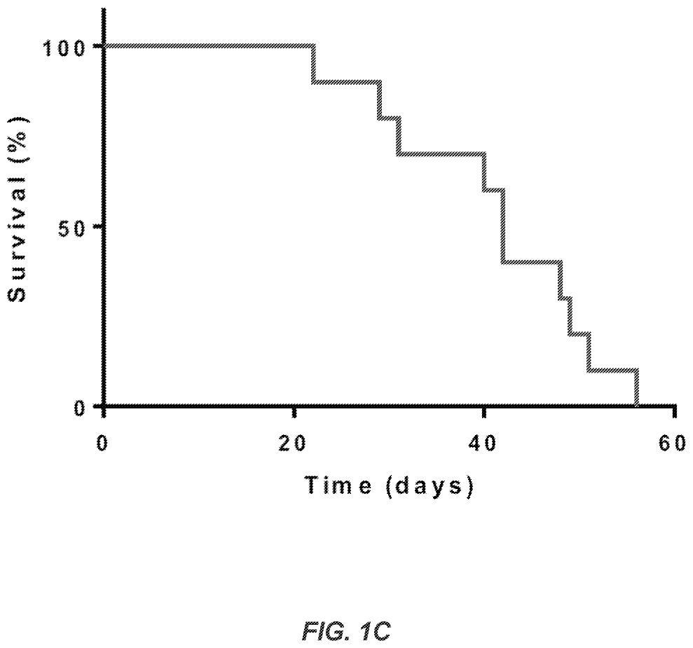

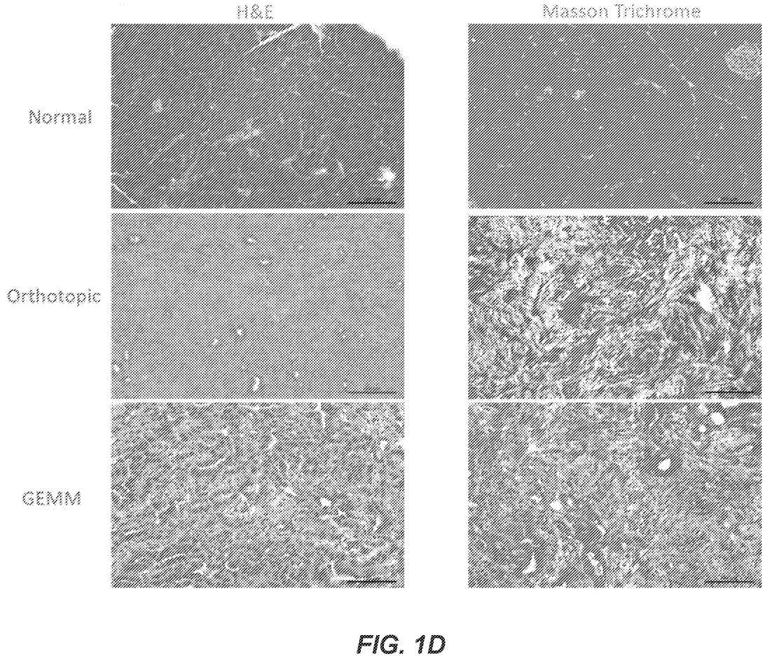

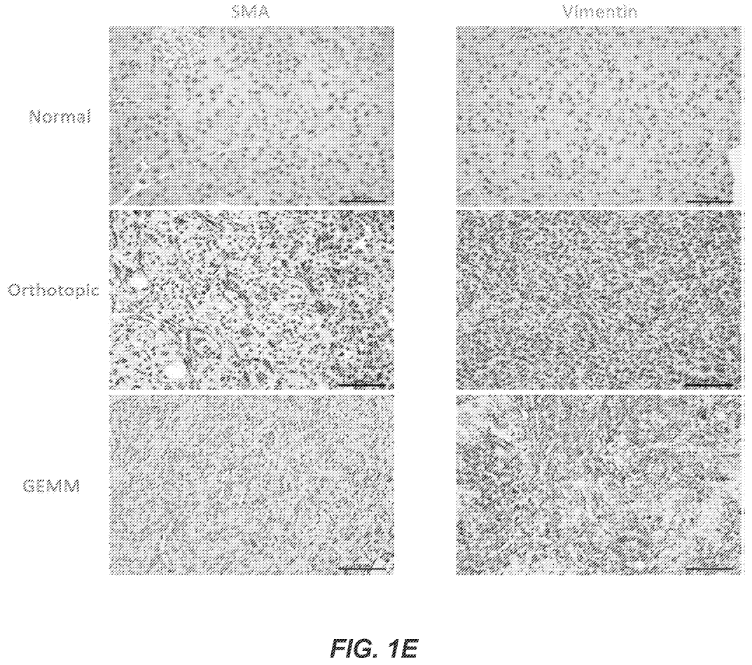

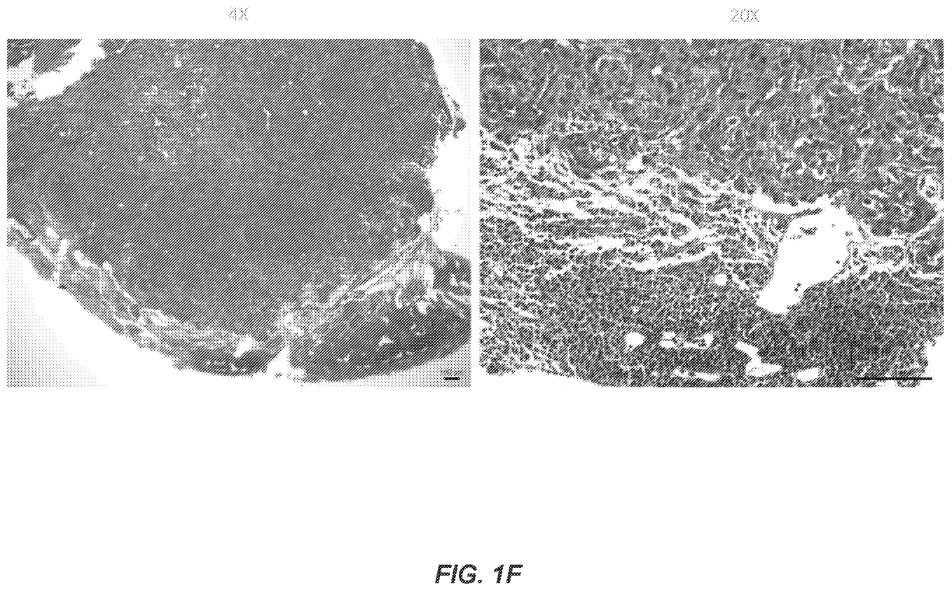

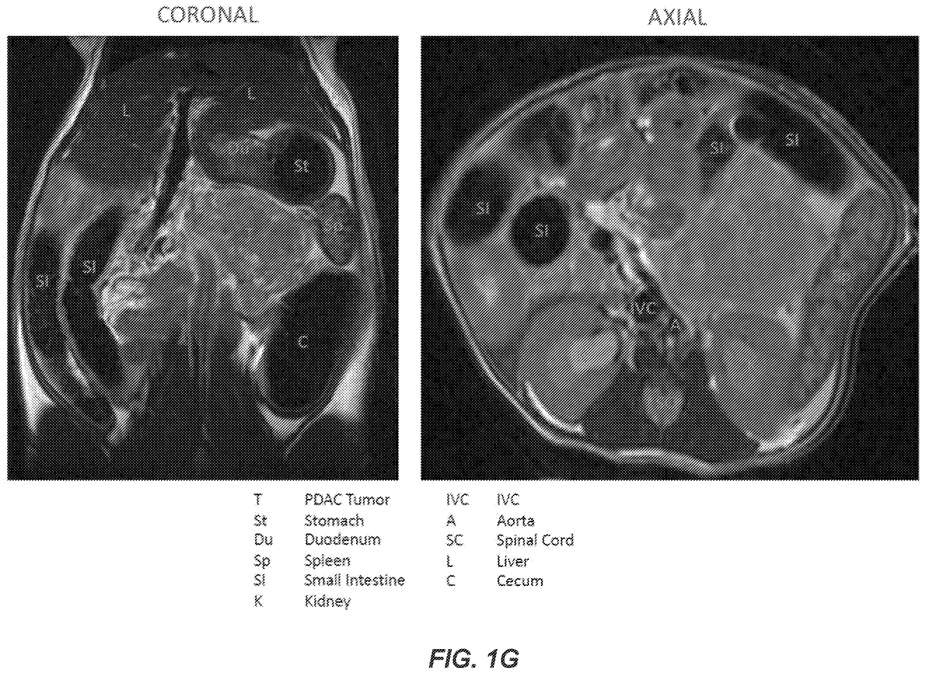

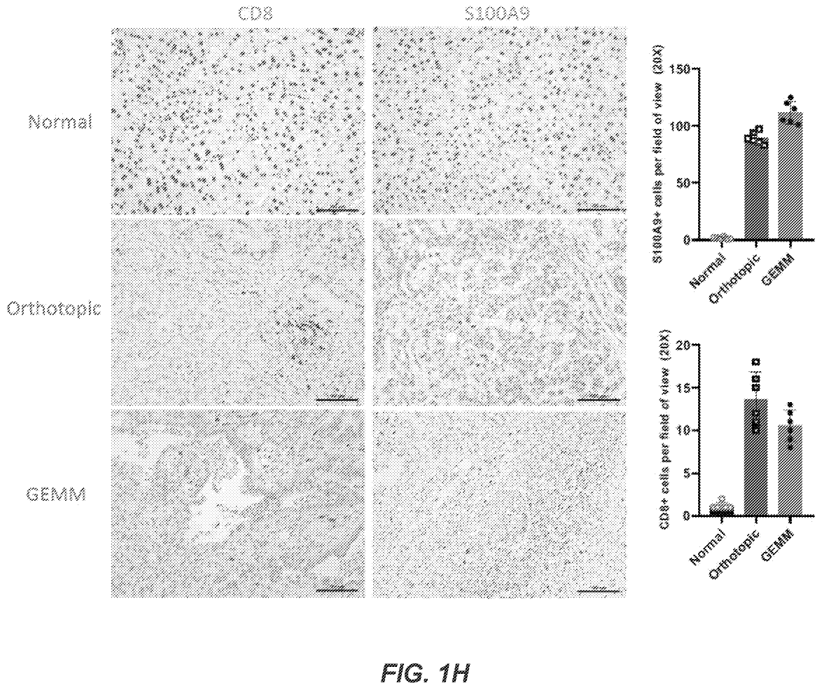

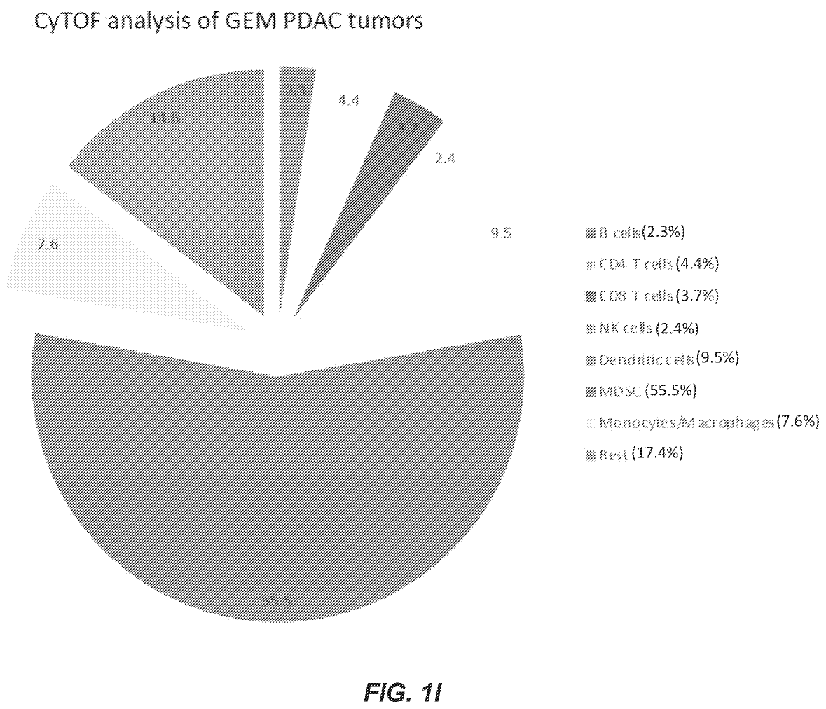

The inducible oncogenic KRAS mouse model (p48Cre; tetO_LSL-Kras^{G12D}; ROSA_rtTA; p53^{L/+}) designated “iKRAS” recapitulates the hallmark features of human PDAC including resistance to all standard therapies used to date [9]. For multi-arm drug testing, iKRAS cell lines were used to generate large cohorts with orthotopic PDAC tumors in syngeneic immunocompetent mice. The tumors grew rapidly to volumes of ˜1000 mm³, demonstrated avid fluorodeoxyglucose (FDG) uptake in 4-5 weeks after implantation, and could be readily detected using PET/CT, MRI and bioluminescence (FIGS. 1A-1B). All mice succumbed to PDAC after 3 to 8 weeks with a median OS of ˜6 weeks (FIG. 1C). Importantly, orthotopic tumors faithfully recapitulated histologic features of autochthonous iKRAS tumors, including glandular tumor structures with poorly differentiated cells, significant desmoplastic response as evidenced by deposition of collagen and high stromal expression of smooth muscle actin (SMA) and vimentin (FIGS. 1D-1E), as well as local invasion into surrounding lymph nodes (FIG. 1F) and organs such as duodenum and kidneys (FIG. 1G).

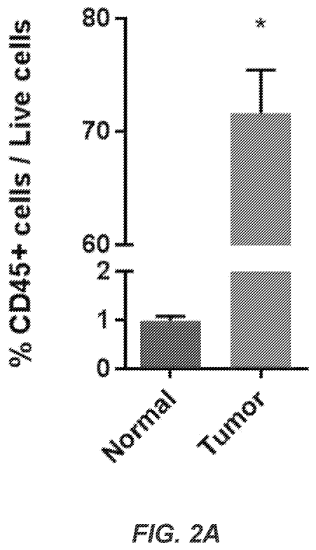

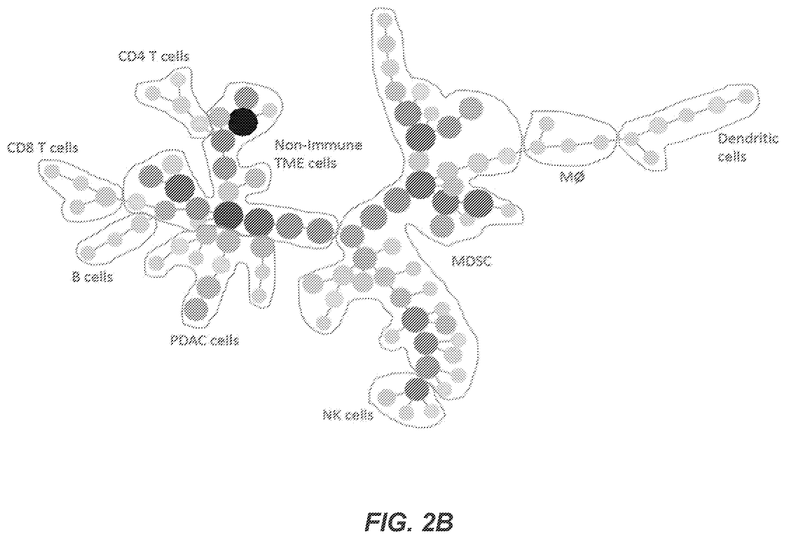

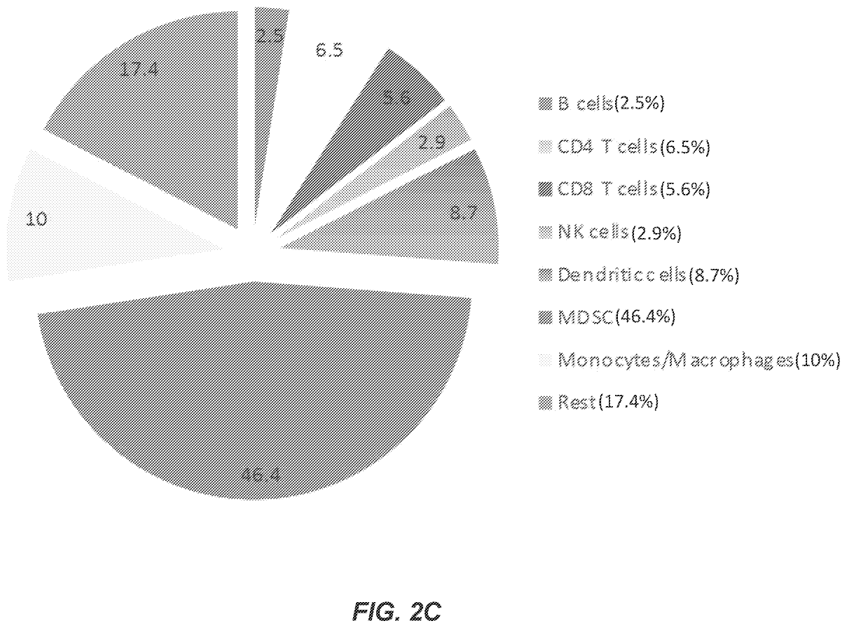

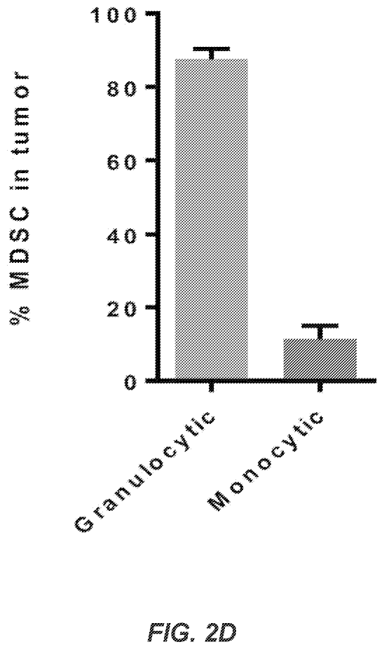

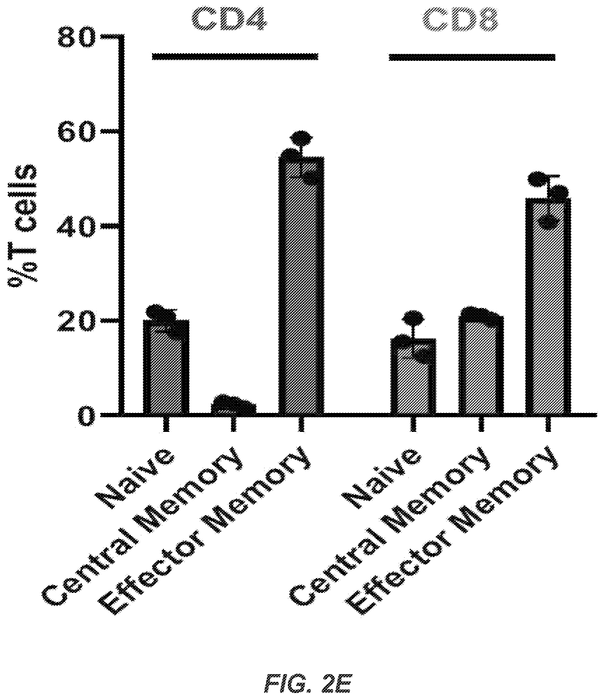

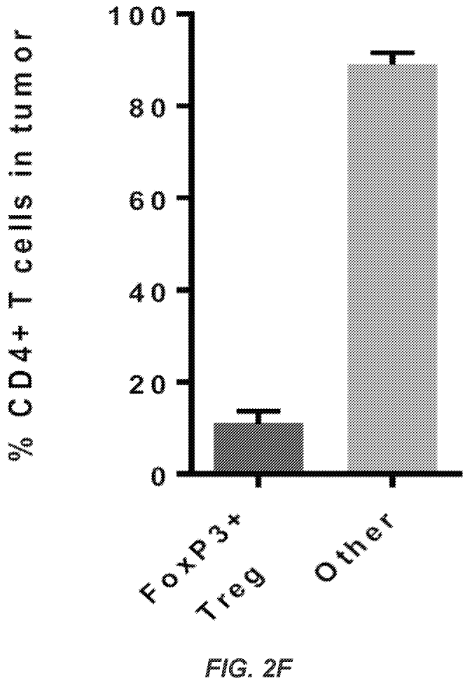

To comprehensively audit the constellation of infiltrating immune cells in the PDAC tumor microenvironment (TME), time-of-flight mass cytometry (CyTOF) was performed on established tumors (4 weeks after initial detection on imaging, tumor volume ˜1000 mm³). CyTOF confirmed a significant increase in CD45⁺-infiltrating immune cells in iKRAS tumors (FIG. 2A). Detailed immunophenotyping of live single cells enabled construction of the spanning-tree progression analysis of density-normalized events (SPADE)-derived tree [10], which is a computational approach to facilitate identification and analysis of heterogeneous cell types (FIG. 2B). SPADE of iKRAS tumors displayed the complexity of the PDAC TME, which were composed of epithelial tumor cells (EpCAM⁺CD45⁻), non-immune TME cells (EpCAM⁻CD45⁻), and infiltrating immune cells (EpCAM⁻CD45⁺) that were further grouped into various subpopulations (FIG. 2C). Within the CD45⁺-infiltrating immune cells, CD11b⁺ myeloid cells, including MDSCs (CD45⁺CD11b⁺Gr1⁺) and TAMs (CD45⁺CD11b⁺Gr1⁻ F4/80⁺) represented a significant proportion of the immune population. The majority of MDSCs within the PDAC TME were neutrophilic/granulocytic in nature (FIG. 2D). CD3⁺ T cells infiltrating PDAC tumors were mostly comprised of CD4⁺ and CD8⁺ effector memory T cells (FIG. 2E). The majority of CD4⁺ T cells were T_{eff} with a small proportion of FoxP3⁺ T_{regs} (FIG. 2F). Contrary to prior hypotheses that desmoplastic stroma in the PDAC TME sequesters T cells in the periphery away from cancer cells, evaluation of the spatial distribution of immune cells by immunohistochemical analysis of orthotopic and autochthonous iKRAS tumors revealed that CD8⁺ T cells can infiltrate the core of PDAC tumors and are found directly adjacent to cancer cells (FIG. 1H). Similarly, S100A9⁺ myeloid cells were identified directly adjacent to cancer cells and CD8⁺ T cells in the core of the tumor (FIG. 1H). The spectrum of immune cells in the TME of autochthonous iKRAS PDAC tumors was compared with those from orthotopic iKRAS PDAC tumors and similar composition and relative proportions of the various immune cells was found (FIG. 1I).

### Example 2—Single-Cell RNA Sequencing Analysis of iKRAS PDAC Tumor-Associated Immune Cells

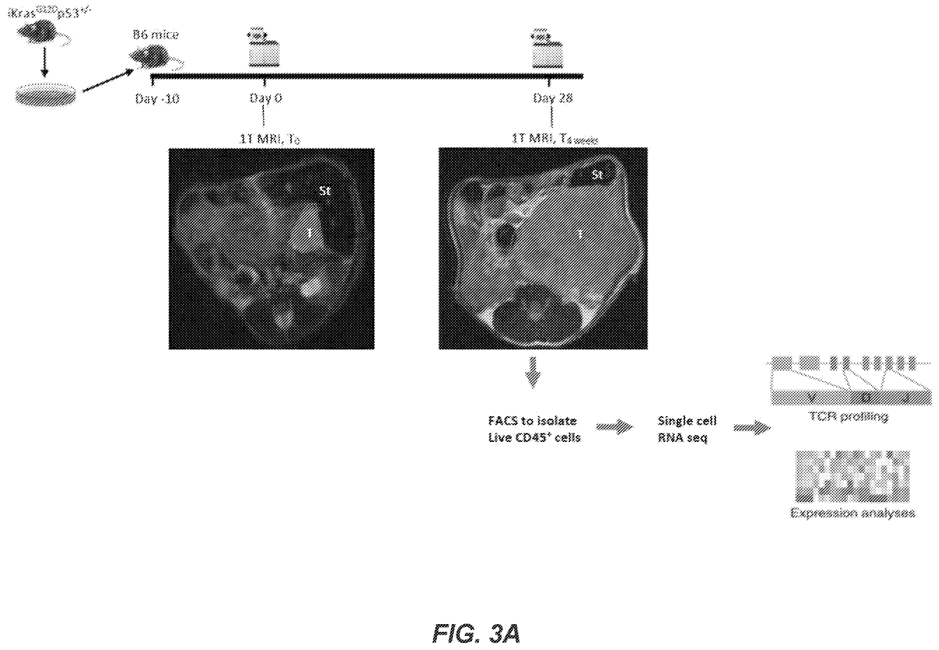

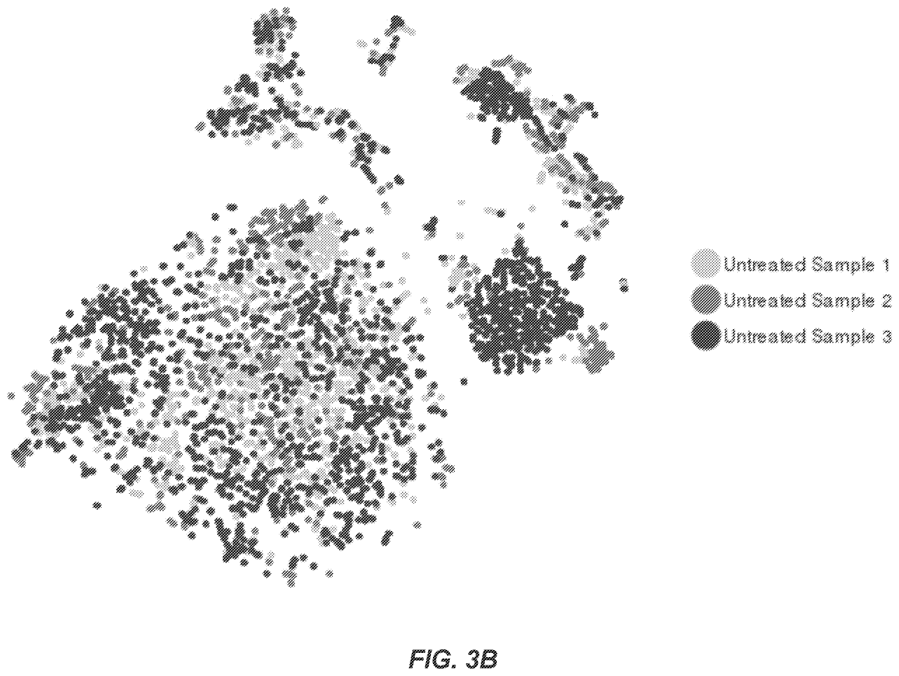

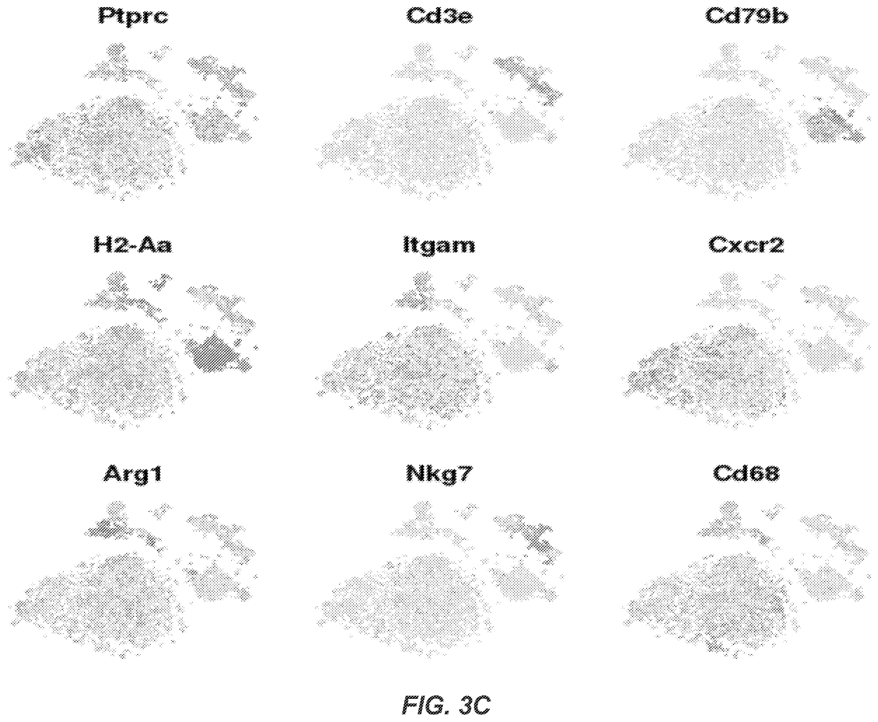

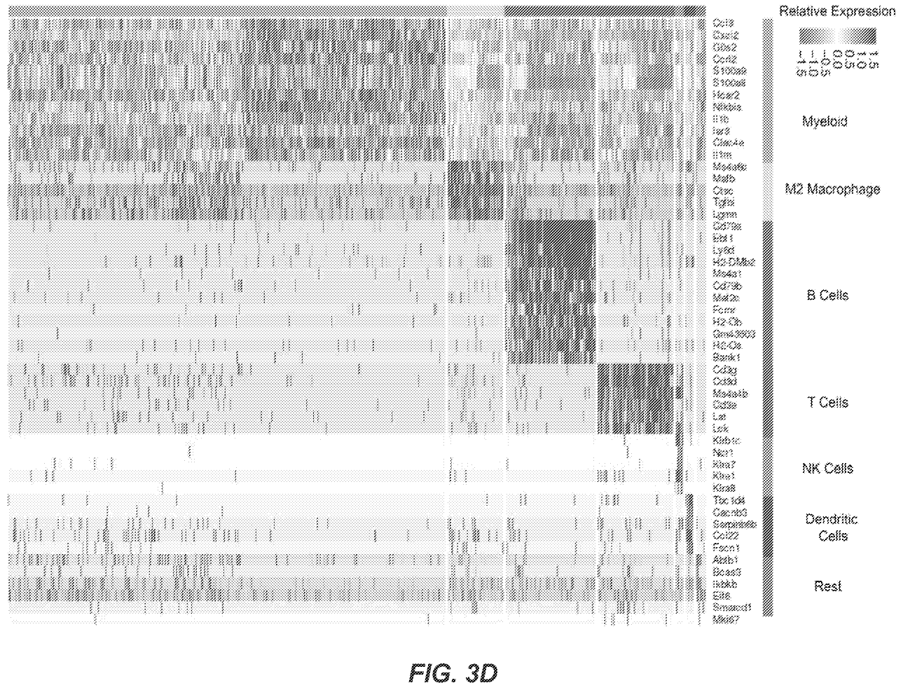

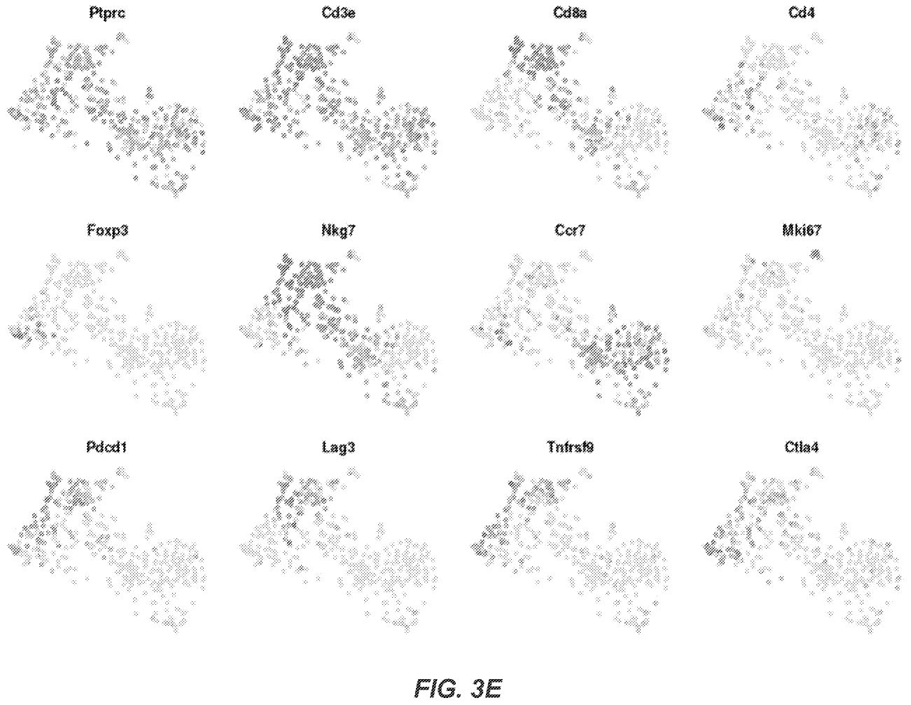

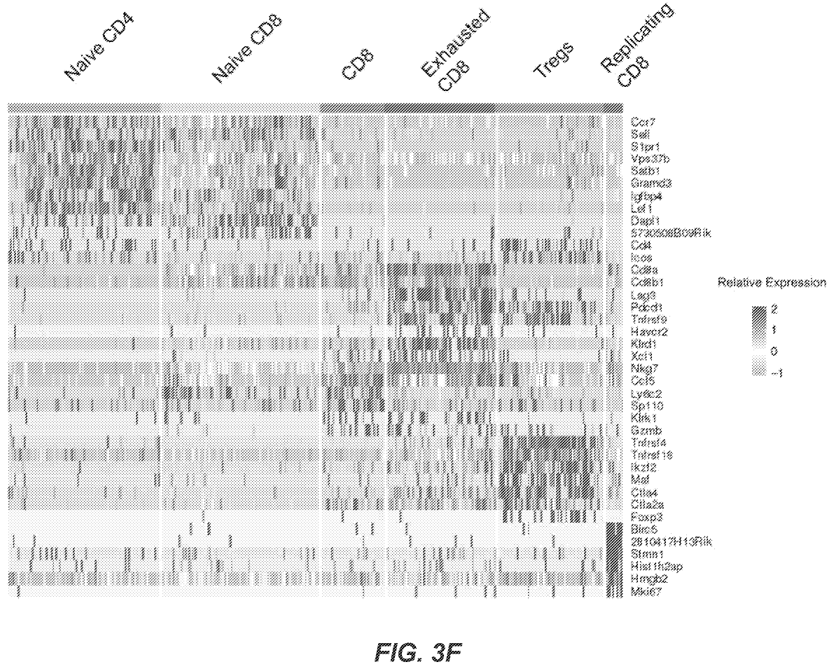

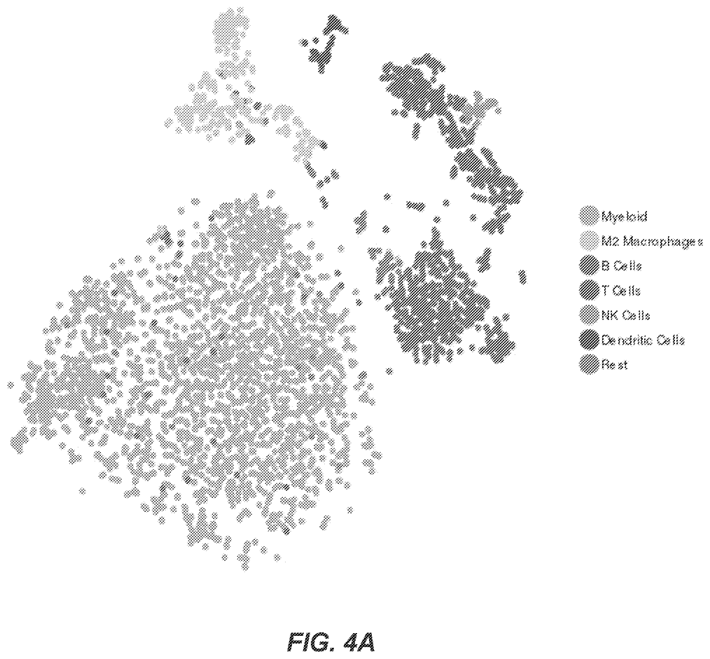

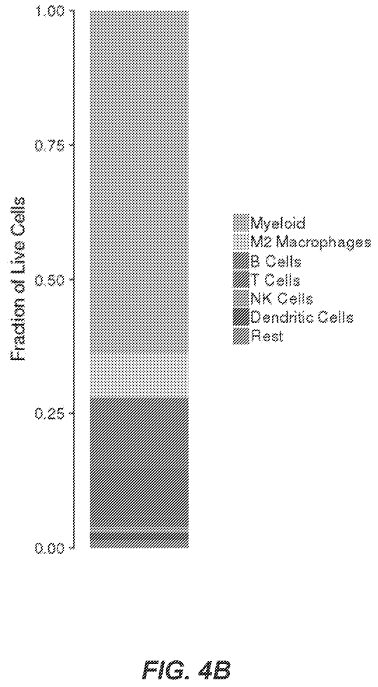

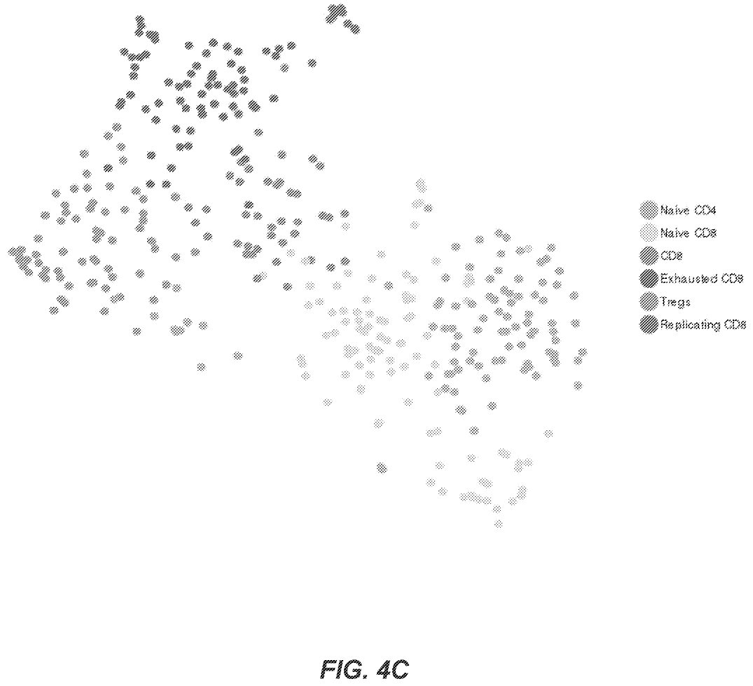

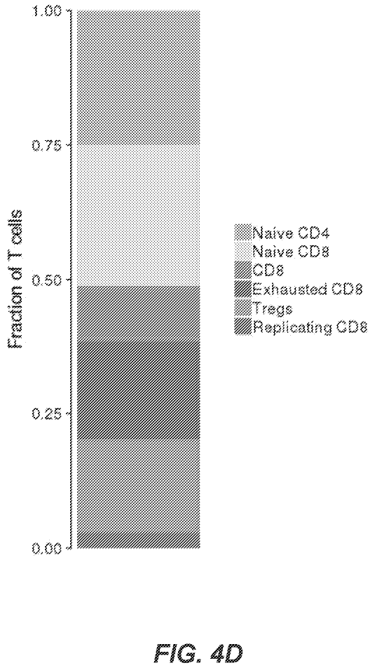

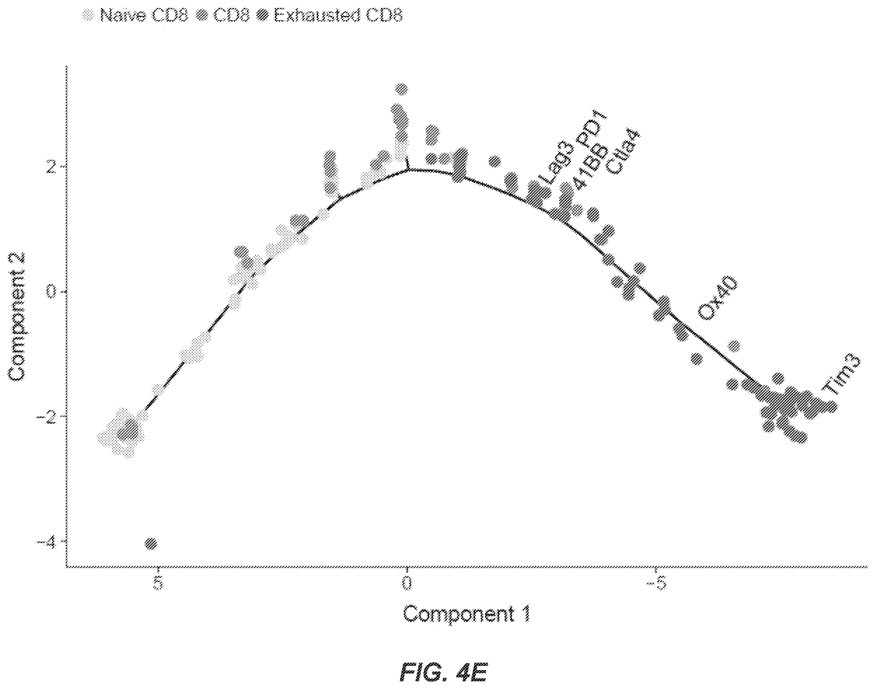

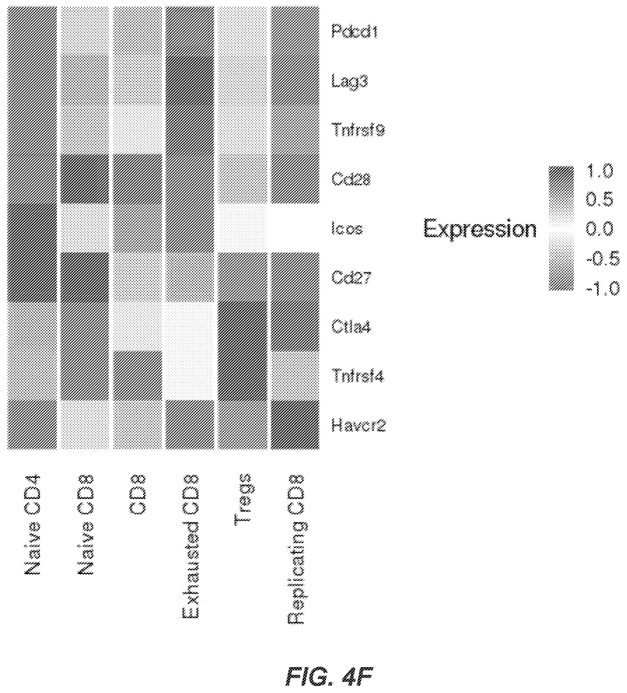

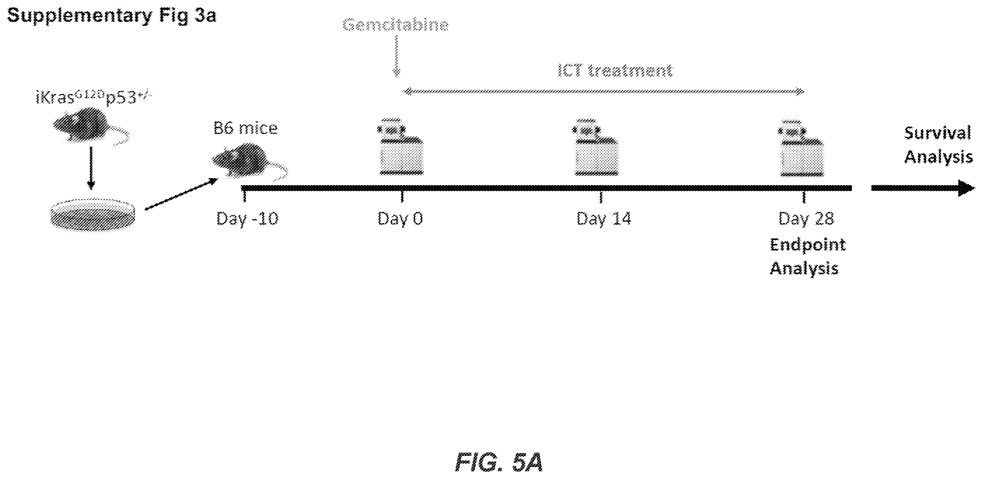

To delineate the complex composition of the immune microenvironment in PDAC, single-cell RNA sequencing was performed on live CD45⁺ immune cells sorted from iKRAS tumors (4 weeks after initial detection on imaging, tumor volume ˜1000 mm³) (FIG. 3A). A total of 4080 sorted individual cells from 3 iKRAS tumors were sequenced to an average of 33,488 confidently-mapped reads per cell (FIG. 3B). Dimensional reduction analysis (t-SNE) and clustering (SNN) applied to the expression data revealed that live CD45⁺ cells clustered into several subgroups (FIG. 4A) with similar fractions (FIG. 4B) as those identified by CyTOF analysis of orthotopic (FIG. 2C) and genetically engineered (FIG. 1I) mouse tumors. The expression of signature genes and known functional markers suggested clusters of immunocytes, including myeloid cells (S100A8/A9 and Cxcr2 expression), M2 macrophages (Mafb and Tgfbi expression), B cells (Cd79b expression), T cells (Cd3 expression), NK cells (Klr expression) and dendritic cells (FIGS. 3C and 3D). The myeloid compartment including MDSCs and M2 macrophages were the predominant immune cells in the PDAC TME.

To reveal the intrinsic heterogeneity and potential functional subtypes of the T cell population, unsupervised clustering was performed using SNN and 6 clusters were identified, including 2 clusters for CD4⁺ and 4 clusters for CD8⁺ T cells (FIGS. 4C and 4D). CD4⁺ T cell clusters included naïve CD4⁺ T cells (Ccr7 and Lef1 expression) and CD4⁺ Tregs (Foxp3 expression as well as Ctla4 and Tnfrsf4 expression) (FIG. 3E). CD8⁺ T cell clusters included naïve CD8⁺ T cells (Ccr7 and Lef1 expression), two separate clusters with expression of cytotoxic genes (Nkg7 and Gzmb), although one of these clusters displayed higher expression of T cell exhaustion markers including Pdcd1, Lag3 and Havcr2 (exhausted CD8⁺ T cells), and a small cluster of highly replicating CD8⁺ T cells (high Ki-67 expression) (FIGS. 3E and 3F). To elucidate the developmental trajectory of CD8⁺ T cells within the PDAC TME, a pseudotemporal time inference algorithm, Monocle2, was applied [13]. Clusters of CD8⁺ T cells formed a linear structure, which when rooted with naïve CD8⁺ T cells, was followed by non-exhausted cytotoxic CD8⁺ T cells and ended with exhausted CD8⁺ T cells (FIG. 4E). Thus, exhausted T cells were highly enriched at the late period of pseudotime, demonstrating the T cell state transition from naïve to activated to exhausted. Expression of activating (Tnfrsf9 and Tnfrsf4) and inhibitory (Pdcd1, Lag3, Ctla4 and Havcr2) immune checkpoints was noted on the exhausted CD8⁺ T cell cluster but not in the naïve or intermediate CD8⁺ T cell states (FIG. 4E) raising the possibility that these molecules may be involved in mediating the exhausted state of CD8⁺ T cells in the PDAC TME. These studies demonstrated preferential enrichment of CD4⁺ Tregs with high Ctla4 and Tnfrsf4 expression as well as exhausted CD8⁺ T cells with high Pdcd1, Lag3, Tnfrsf9 and Havcr2 expression amongst the differentiated T cell population in PDAC (FIG. 4F). The expression of these checkpoints on CD4⁺ and CD8⁺ T cells was validated using flow cytometry. Since these two T cell subsets are targets for cancer immunotherapies [2, 4, 8], further analyses focused on the role of these hitherto uncharacterized novel immune checkpoints in PDAC identified to be expressed on these cells.

### Example 3—Analysis of Immune Checkpoint Therapy in PDAC

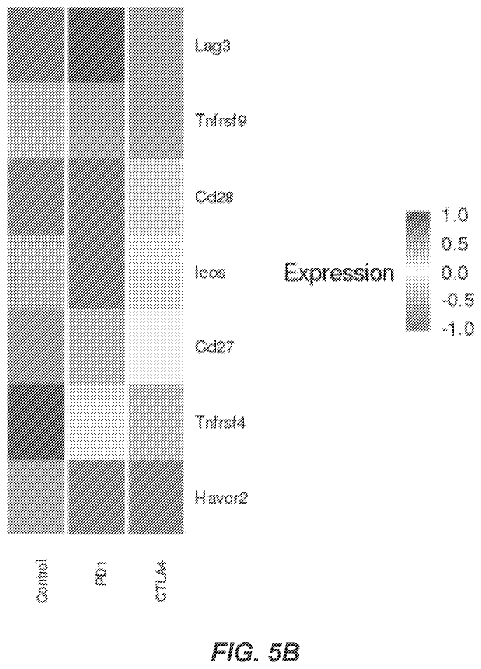

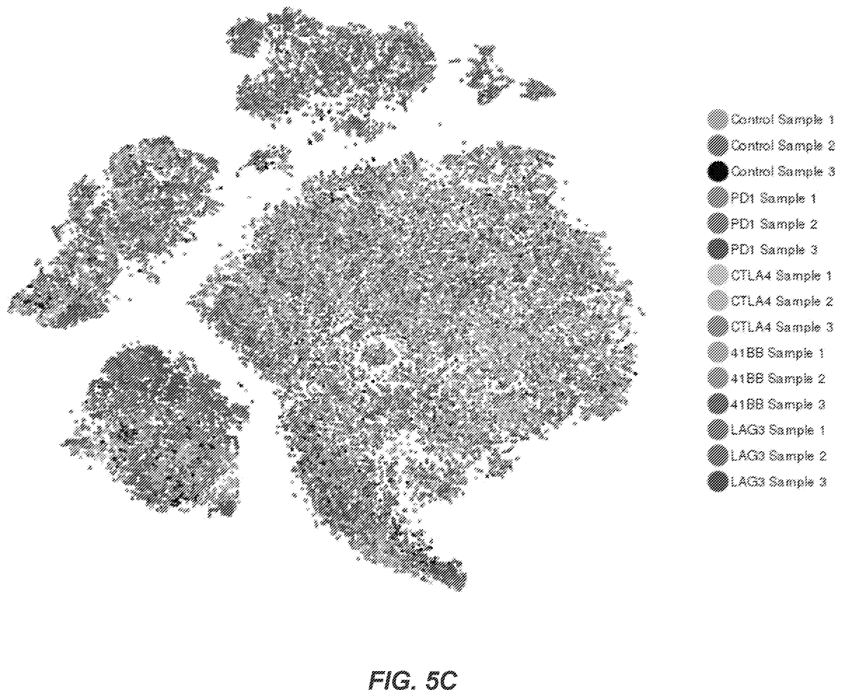

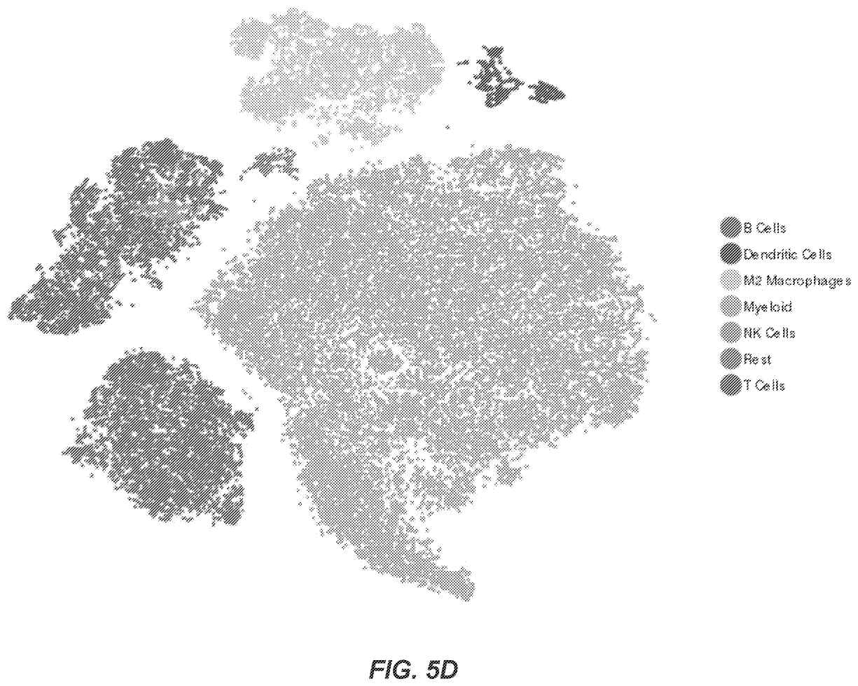

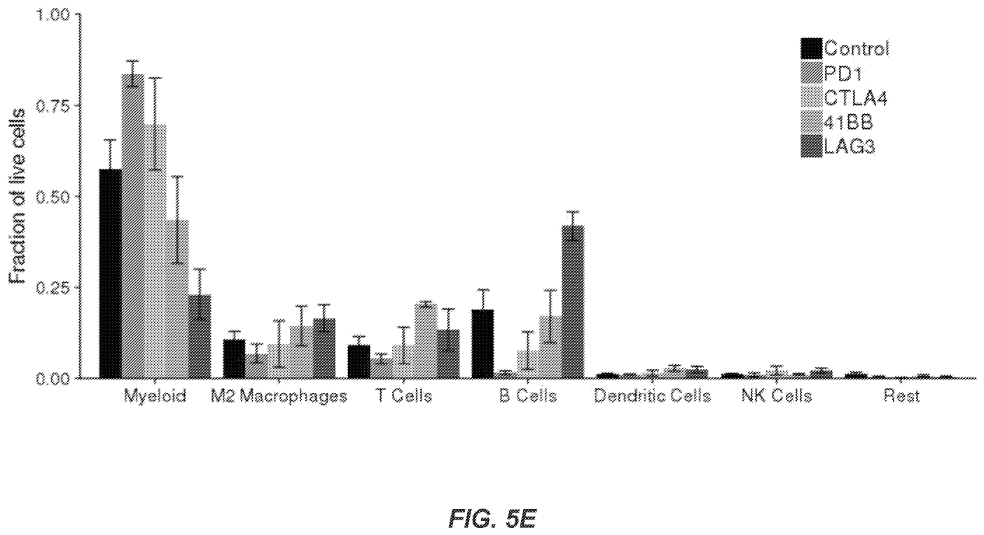

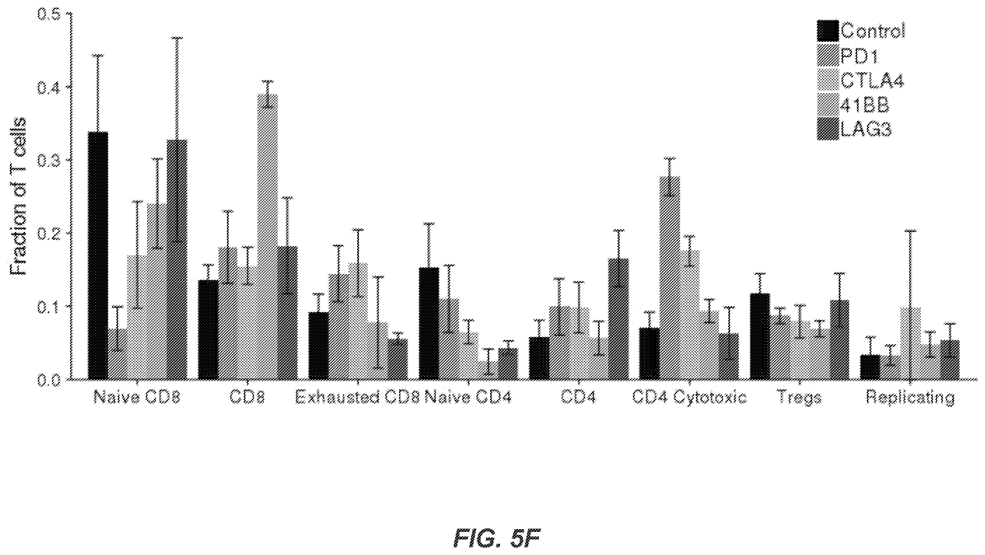

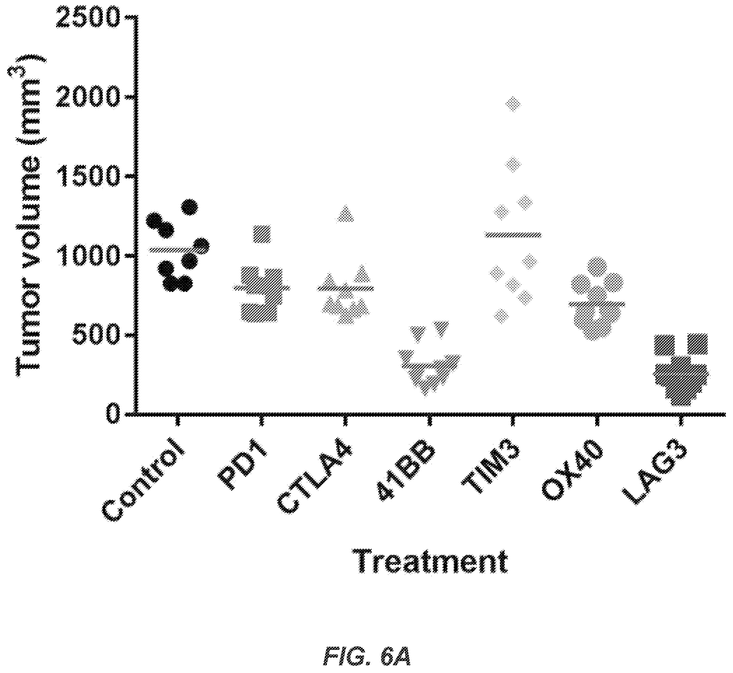

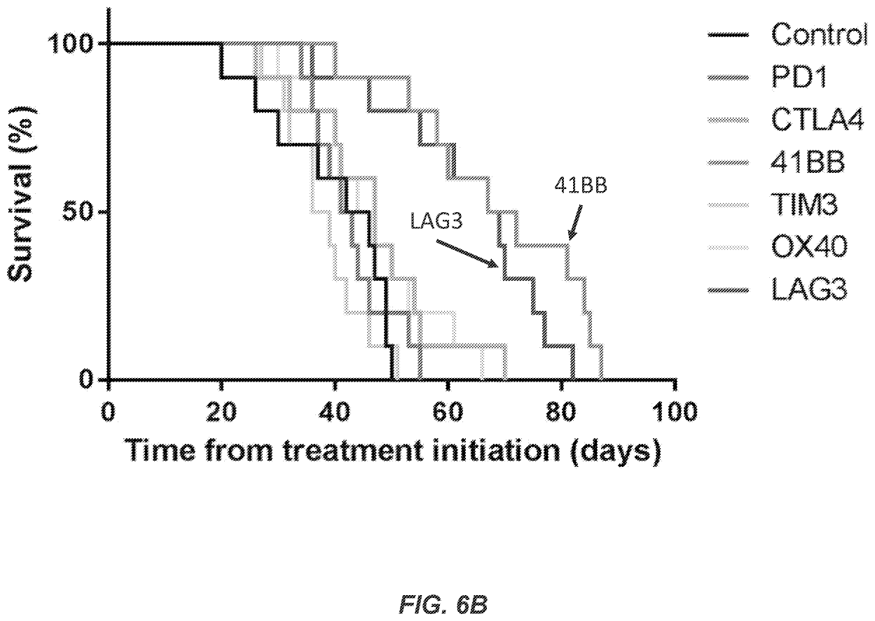

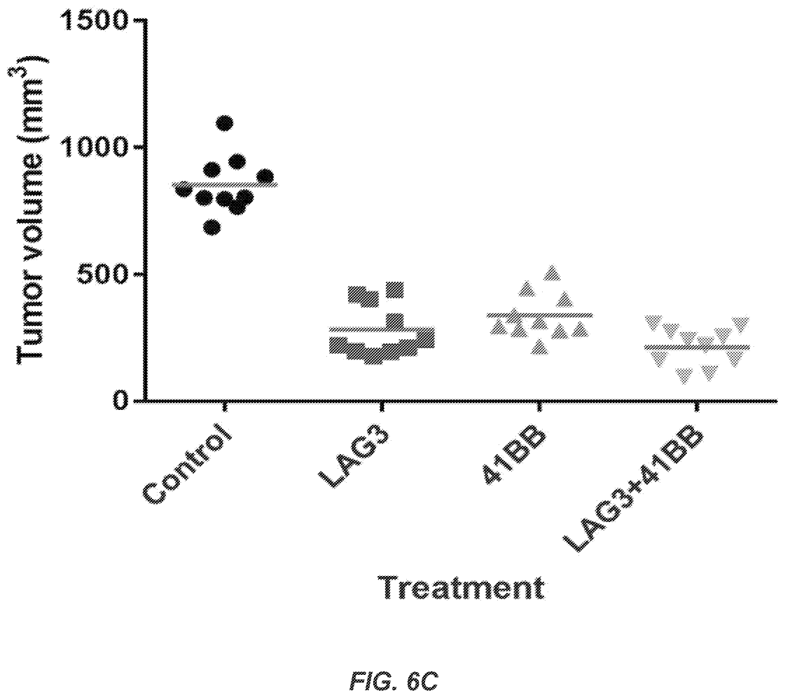

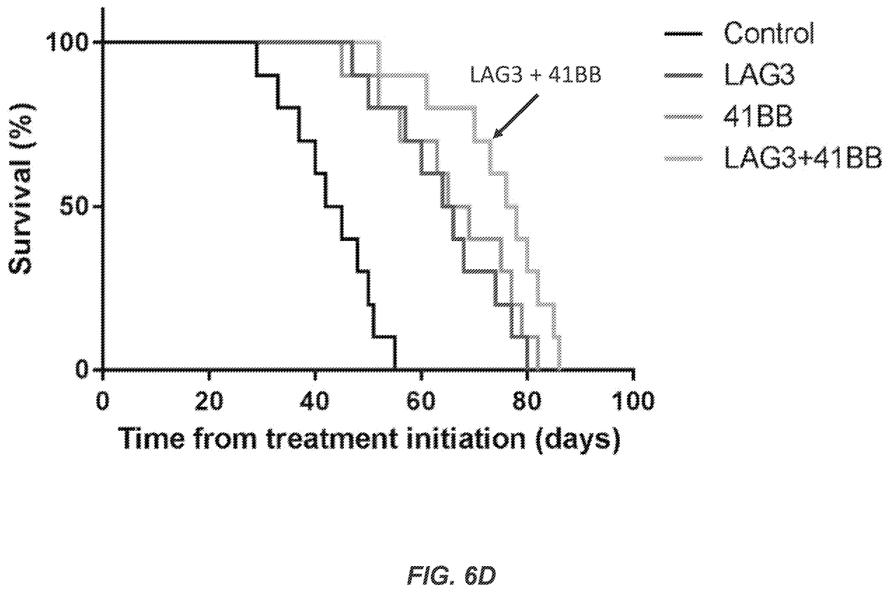

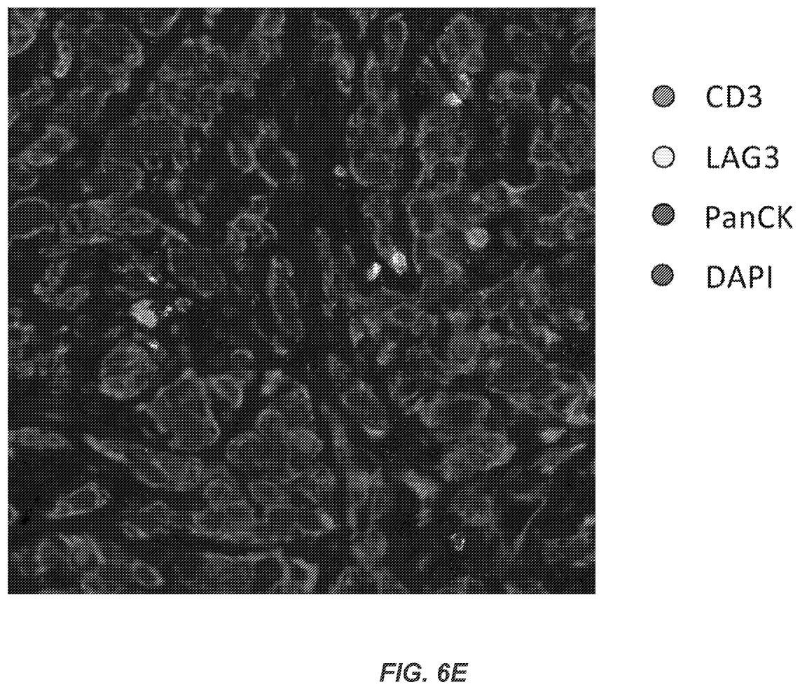

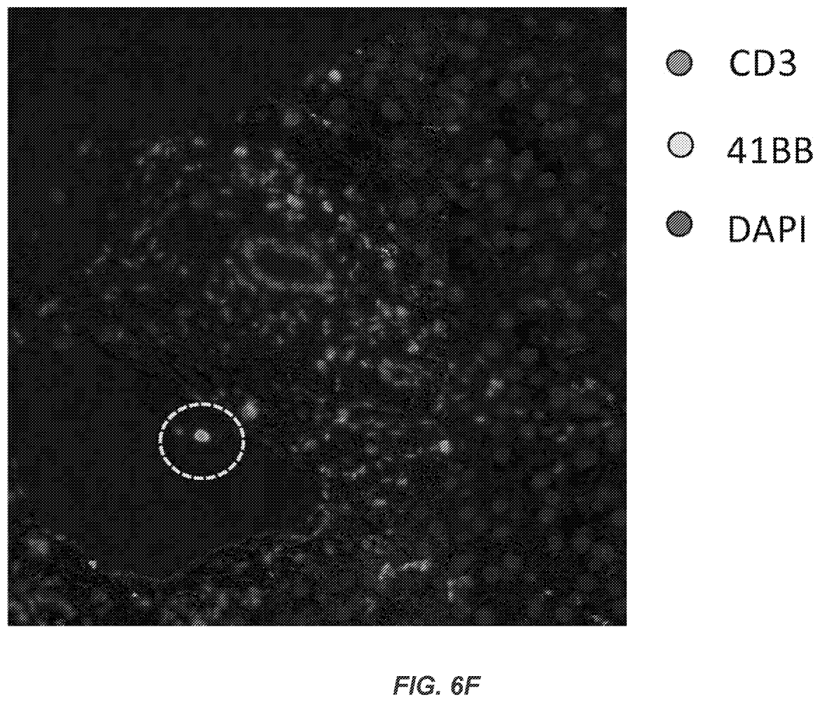

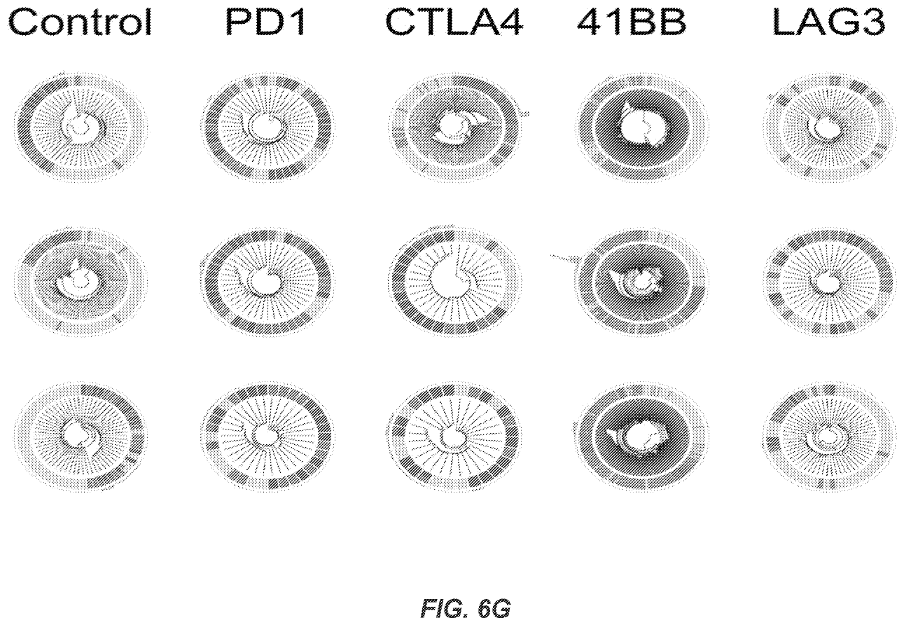

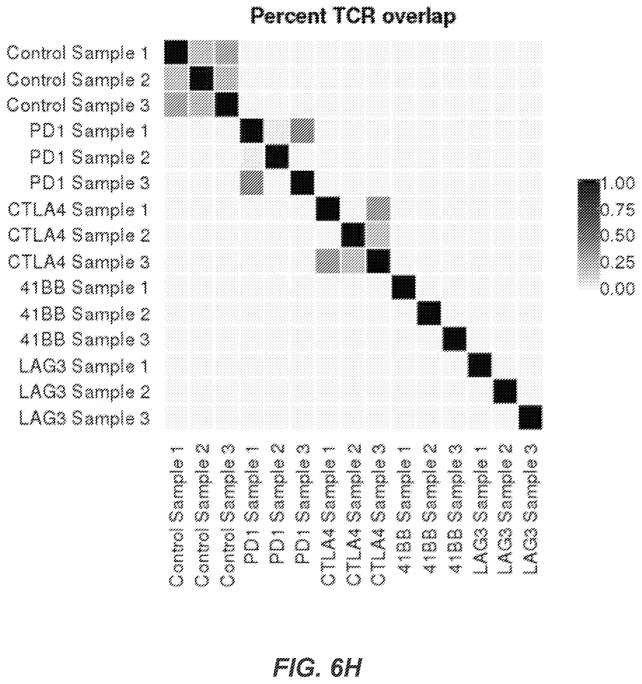

The presence of intra-tumoral CD8⁺ T cells as demonstrated in both autochthonous and orthotopic mouse models (FIG. 2C, FIG. 1I, FIGS. 4A-4D), taken together with recent studies in human PDAC which found that increased infiltration of CD8⁺ T cells is associated with improved OS of patients [11, 12], suggested the presence of an active immune response with the potential for immunotherapeutic approaches targeting PDAC. However, the efficacy of single-agent ICT in treatment of PDAC patients thus far has been underwhelming, contributing to the perception that PDAC is non-immunogenic [4, 5, 6, 7]. To corroborate this hypothesis, immunocompetent C57BL/6 mice with PDAC orthotopic tumors were enlisted into multi-armed therapeutic trials of various agonist and antagonist ICT antibodies targeting the aforementioned immune checkpoints with high expression on differentiated T cells in iKRAS PDAC tumors (FIG. 5A). Mice with MRI-documented PDAC tumors of equivalent size were treated with a single dose of gemcitabine and were subsequently randomized to receive single or combination ICT treatments for 4 weeks before endpoint analysis (FIG. 5A). Induction with a single dose of gemcitabine chemotherapy was used given: (1) known anti-tumor effects of gemcitabine against PDAC, based on its use in chemotherapy regimens for human PDAC patients, which would lead to tumor cell death and increased antigenicity/antigen presentation by dendritic cells in the TME, and (2) known ability to decrease MDSC/Treg accumulation and activity in murine models and human PDAC specimens [3, 4, 14, 15, 16]. Similar to previous clinical trials in PDAC patients [5, 6, 7], it was found that antagonist PD1 and CTLA4 antibodies did not have any appreciable effect on tumor growth or OS of PDAC bearing mice (FIGS. 6A and 6B). Although it was previously believed that PDAC tumors do not respond to anti-CTLA4 and anti-PD1 ICT due to poor infiltration of effector T cells, it was found that despite infiltration of CD4⁺ and CD8⁺ T cells with high Ctla4 and Pdcd1 expression into iKRAS PDAC tumors, respectively, these antibodies are unable to induce effective antitumor immunity (FIGS. 6A and 6B). Evaluating expression of alternate immune checkpoints on T cells isolated from anti-PD1 or anti-CTLA4 treatment-resistant PDAC bearing mice, increased Lag3 and Havcr2 (TIM3) expression was identified in response to anti-PD1 and anti-CTLA4 monotherapy as well as increased Tnfrsf9 (41BB) expression in response to anti-CTLA4 treatment (FIG. 5B). Conversely, decreased expression of Tnfrsf4 (OX-40) was noted in response to anti-PD1 and anti-CTLA4 monotherapy. Given the relative change in expression of these immune checkpoints which are highly expressed on CD4⁺ Tregs and exhausted CD8⁺ T cells in iKRAS tumors with purported immunomodulatory activity related to potential to enhance or negate T cell activity, the effects of treatment with agonist (41BB and OX40) and antagonist (LAG3 and TIM3) antibodies as monotherapy were evaluated in this model. In contrast to PD1 and CTLA4, monotherapy with agonist 41BB (clone LOB12.3; BioXCell BE0169) and antagonist LAG3 (clone C9B7W; BioXCell BE0174) antibodies attenuated PDAC progression with corresponding increase in OS of PDAC bearing mice (FIGS. 6A and 6B). Given these findings, dual ICT with combination of agonist 41BB and antagonist LAG3 antibodies was evaluated (FIGS. 6C and 6D). Treatment with dual ICT was well tolerated with no deaths during the four week treatment period. A modest increase in efficacy with reduced progression, and increased survival with combination compared to monotherapy, was found; however all mice eventually succumbed. Together, these findings suggested that activation of T cell activity with agonist 41BB and/or antagonist LAG3 antibodies is necessary for robust growth inhibition of iKRAS PDAC tumors but not sufficient to induce complete elimination of established tumors. To corroborate the translational potential of these findings, the expression of these novel immune checkpoint targets was validated in human PDAC using multiplex IHC (FIGS. 6E and 6F).

To evaluate dynamic changes in the immune microenvironment with the various ICT treatments, single-cell RNA sequencing was performed on live CD45⁺ immunocytes sorted from PDAC bearing mice treated with effective (agonist 41BB and antagonist LAG3) and ineffective (antagonist PD1 and CTLA4) ICT agents after a 4-week period (n=3 per treatment group). A total of 45,533 cells were sequenced to an average depth of 29,290 confidently-mapped reads per cell (FIG. 5C). Dimensional reduction analysis (t-SNE) applied to the expression data revealed that immune cells clustered into similar subtypes of immune cells as untreated tumors with the exception of cytotoxic CD4⁺ T cells (characterized by Gzmk expression) (FIG. 5D). A relative decrease was identified in the proportion of immunosuppressive myeloid cells with effective (antagonist LAG3 and agonist 41BB) antibody treatment and an increase was identified with ineffective (antagonist PD1 and CTLA4) antibody treatment (FIG. 5E). Treatment with agonist 41BB antibody resulted in expansion of T cells (predominated by non-exhausted cytotoxic CD8⁺ T cell clusters) while antagonist LAG3 antibody treatment resulted in relative expansion of CD4⁺ T cells in the immune infiltrate of iKRAS PDAC tumors (FIGS. 5E and 5F). Also identified was a relative increase in the proportion of antigen-presenting dendritic cells with effective (antagonist LAG3 and agonist 41BB) but not ineffective (antagonist PD1 and CTLA4) antibody treatments (FIG. 5E).

To determine the lineage of each individual T cell in the PDAC TME after the various ICT treatments, primers were designed for the mouse a and R TCR locus and targeted PCR performed on the 10×genomics single-cell 5′ cDNA product. From the TCR product library, the full-length TCR α and β sequences were assembled using the 10×genomics cellranger vdj pipeline (FIG. 6G). Overall, 41BB treatment resulted in the most CD8⁺ cell clonotype expansion, comprised mainly of non-exhausted cytotoxic T cells. The overlap in TCR CDR3 sequences from T cells was further evaluated and it was found that anti-PD1 and anti-CTLA4 treated mice harbored significant overlap amongst TCRs between mice within their treatment group, similar to the control (IgG) treatment group. Meanwhile, mice treated with agonist 41BB and antagonist LAG3 antibodies exhibited complete loss of TCR overlap, suggestive of diversification of TCR repertoire (FIG. 6H).

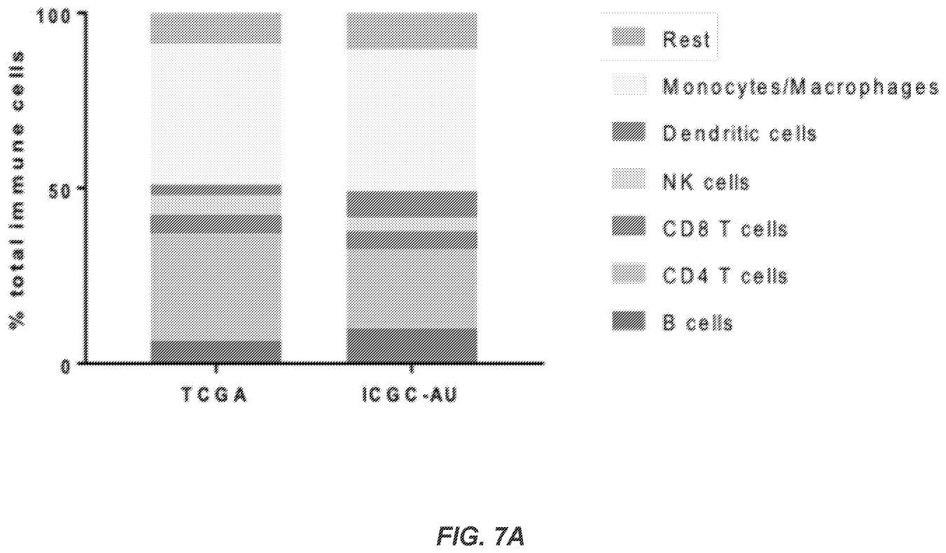

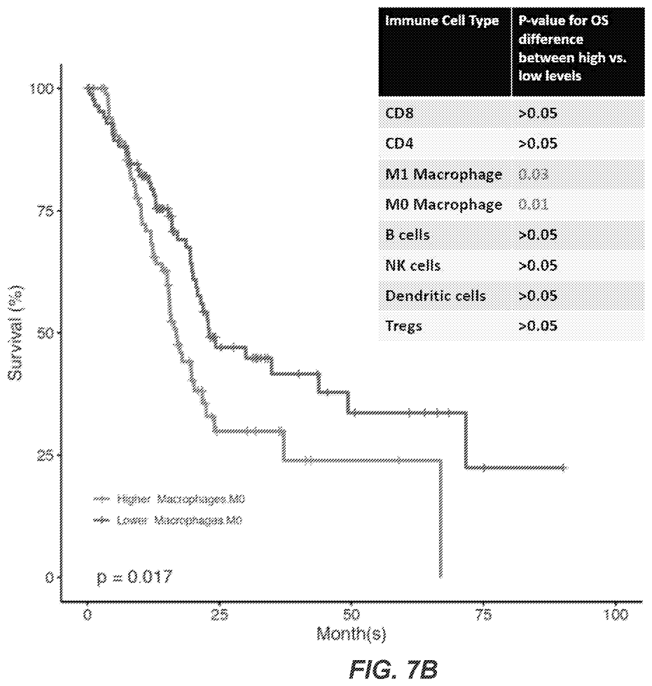

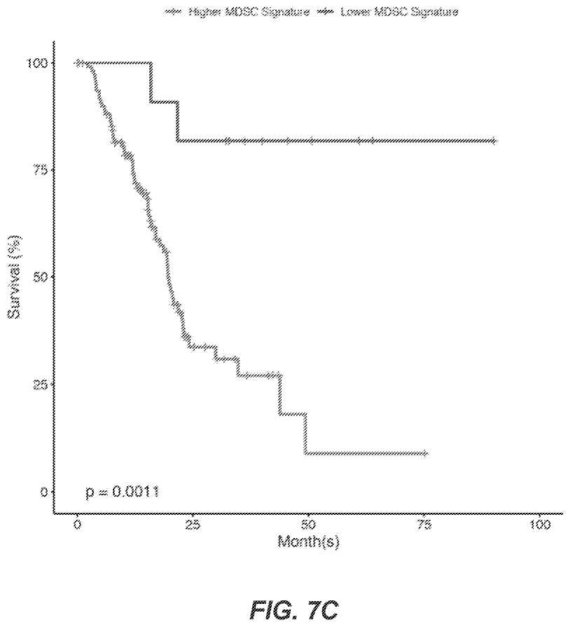

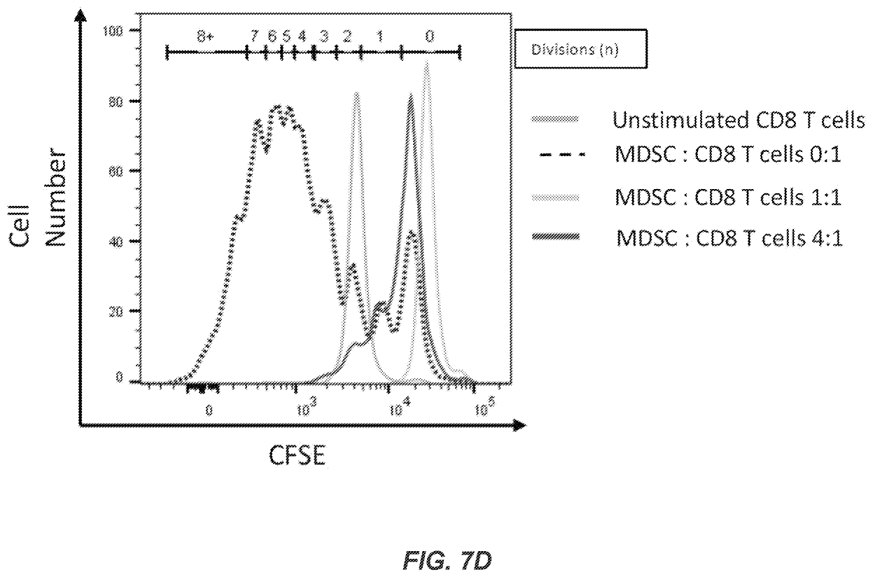

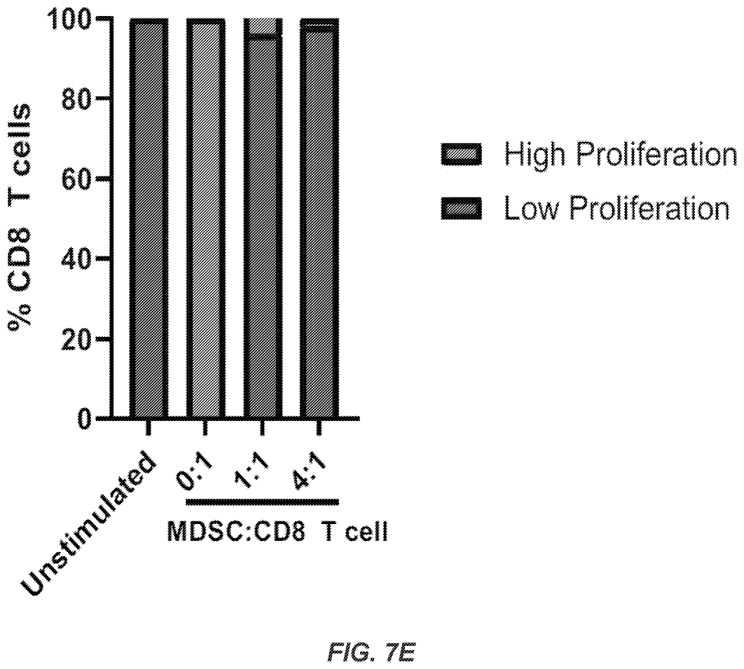

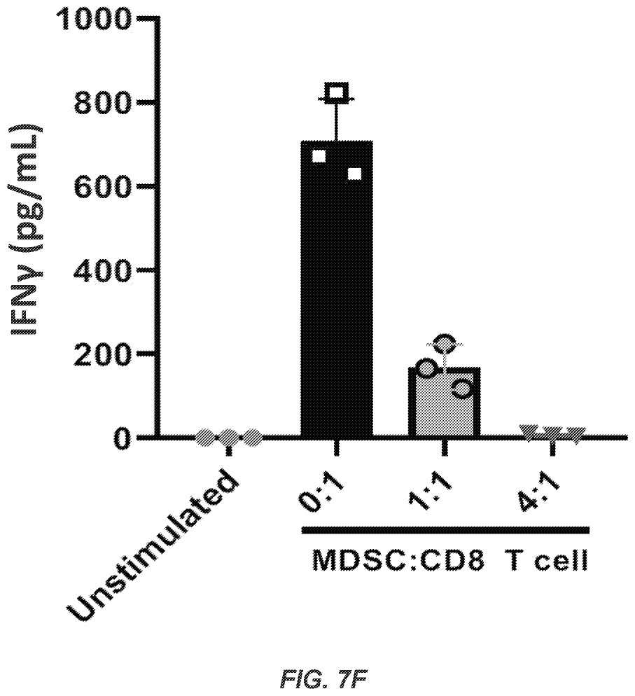

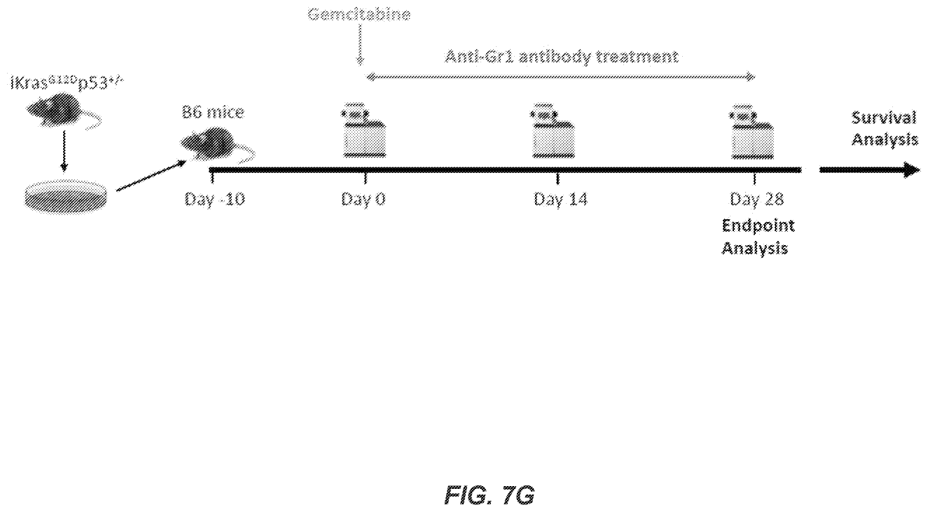

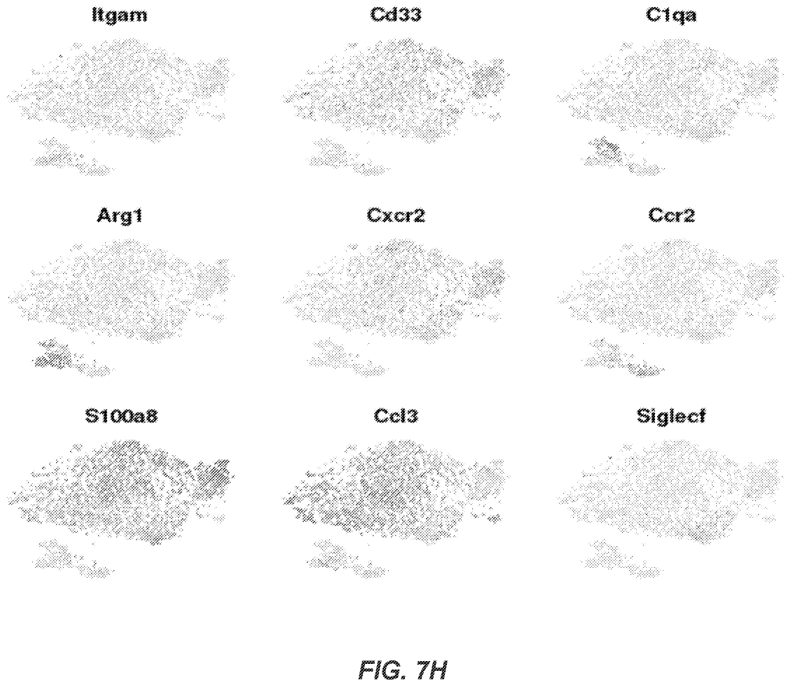

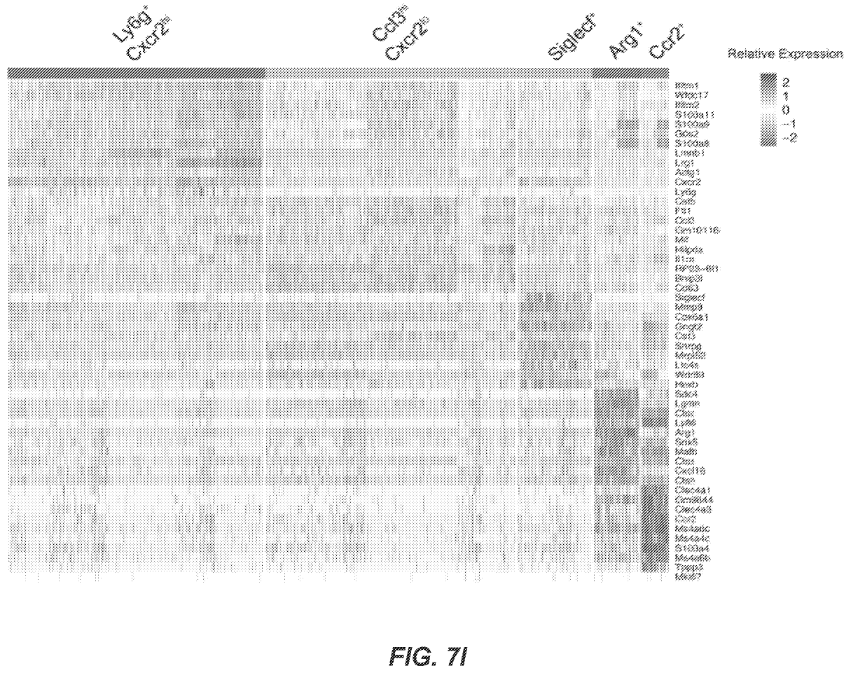

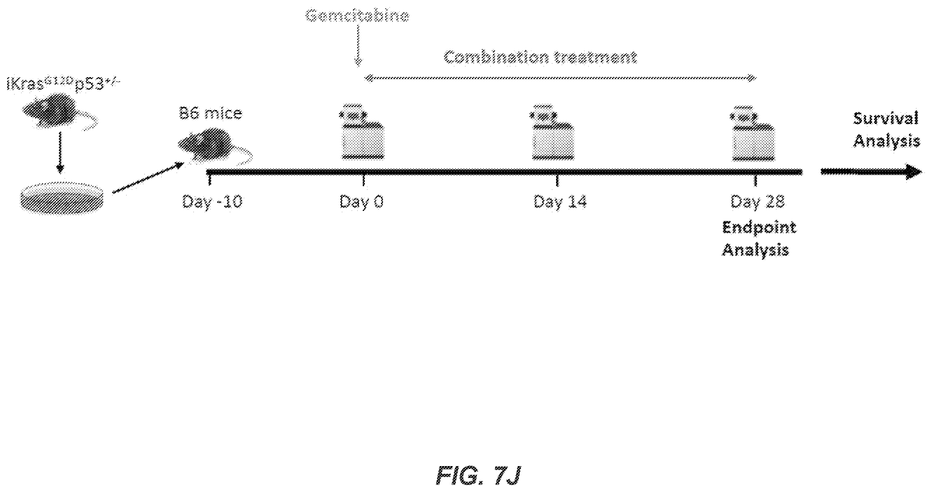

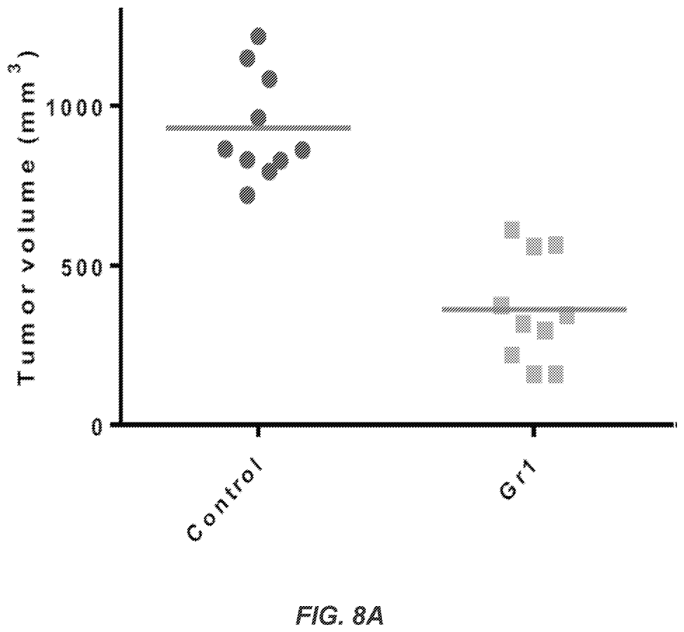

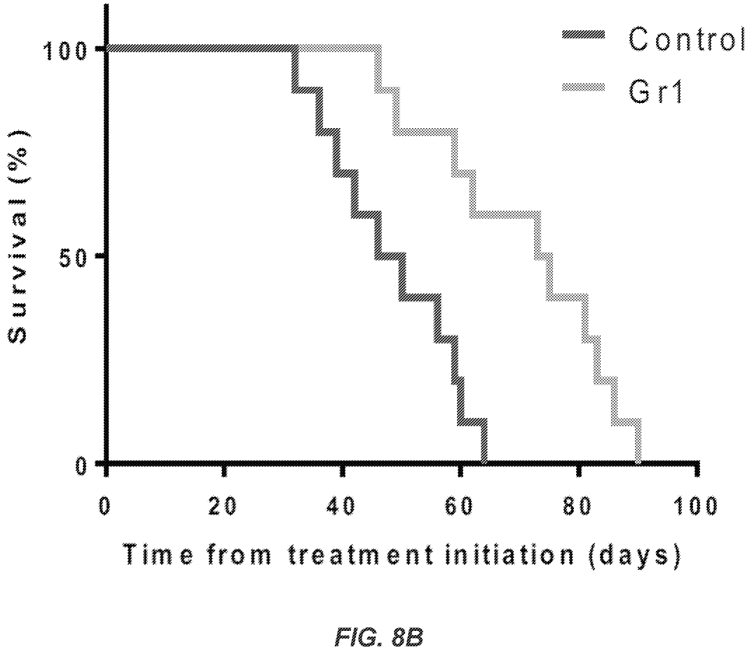

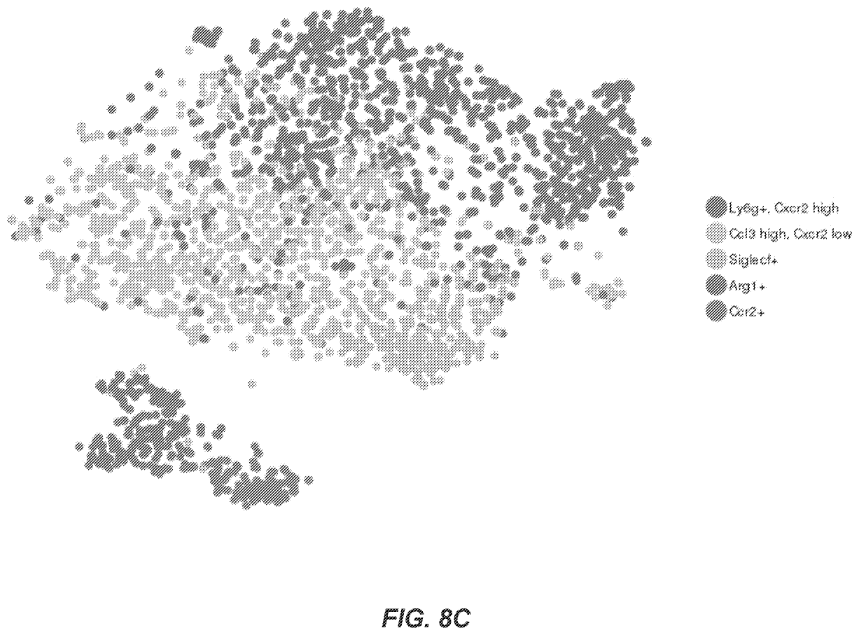

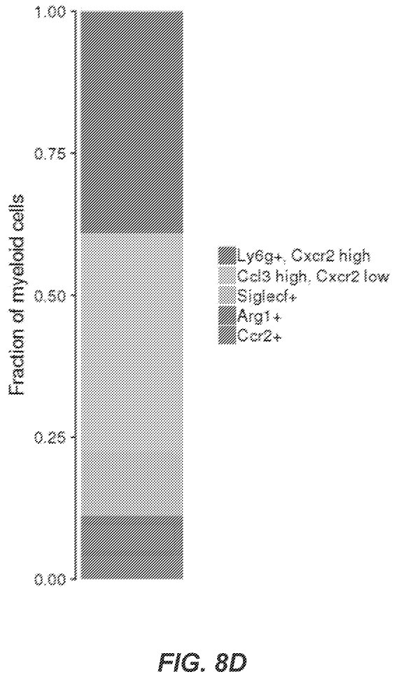

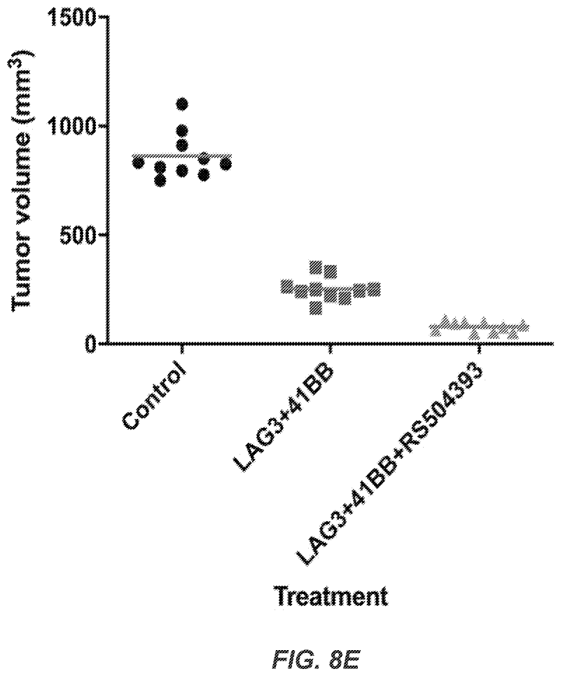

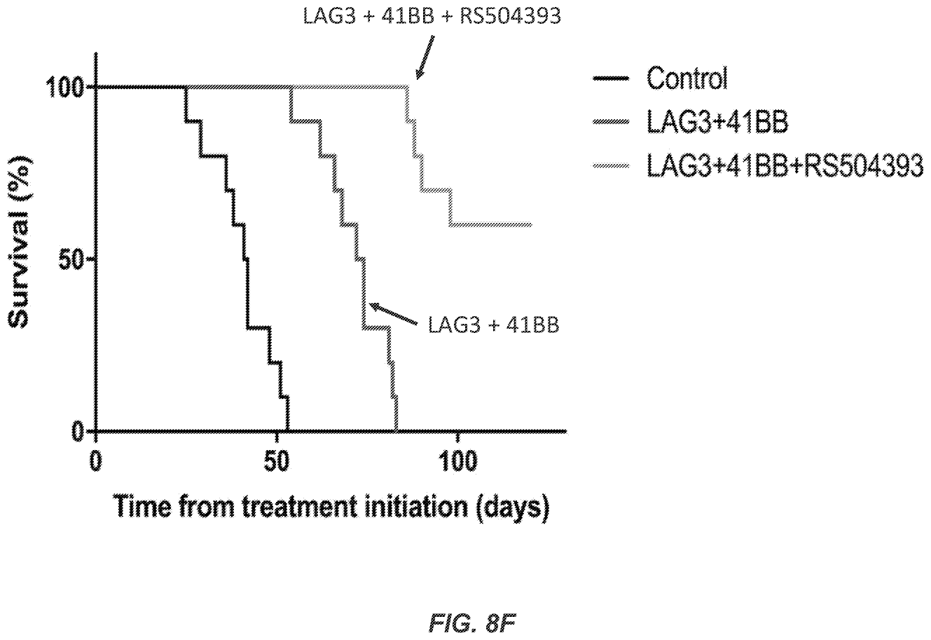

In order to evaluate the physiological significance of myeloid cell types in human PDAC biology, the CIBERSORT deconvolution algorithm was applied to PDAC TCGA and ICGC-AU cohorts to enumerate fractions of immune cell subsets [17]. Consistent with iKRAS tumors, monocytes/macrophages were found to be the predominant immune cell subtype present in both cohorts of human PDAC (FIG. 7A). Amongst the macrophages, the predominant cell type in both TCGA and ICGC-AU cohorts were M2 macrophages. It should be noted that this algorithm does not allow deconvolution of MDSCs from other myeloid cell subtypes, such as monocytes/macrophages. Evaluated next was the impact of each immune cell gene signature on OS of PDAC patients [10, 17]. It was found that macrophage representation correlated significantly with OS of PDAC patients—specifically, those with higher M0 and M1 macrophage signature showed worse overall prognosis (FIG. 7B). These findings were validated in the PDAC ICGC-AU cohort with similar results. Since this algorithm does not allow deconvolution of MDSCs from other myeloid cell subtypes, unsupervised clustering of TCGA PDAC RNA-sequencing data was performed with a 39-gene MDSC signature [18] and 178 TCGA primary PDAC tumors were categorized into three subtypes: MDSC-high (n=114), MDSC-medium (n=54), and MDSC-low (n=11), suggesting that a large proportion of human PDAC tumors may have prominent infiltration of MDSCs (FIG. 7C). Patients with higher MDSC gene expression had significantly lower OS compared to those with lower MDSC expression. Prior studies have demonstrated increased frequency of MDSCs in the bone marrow and peripheral circulation of PDAC patients, which correlates with disease stage [19]. Enrichment of MDSCs in autochthonous and orthotopic iKRAS PDAC tumors (FIG. 2C, FIG. 1I), together with the aforementioned human TCGA PDAC analyses (FIG. 7C), prompted the exploration of the role of MDSCs in iKRAS PDAC tumor progression. To evaluate the potential immunosuppressive activity of intratumoral CD11b⁺Gr1⁺ cells from iKRAS tumors, T-cell proliferation was examined using a standard cell coculture system. These CD11b⁺Gr1⁺ cells strongly suppressed CD3 and CD28 antibody-induced T-cell proliferation and activation (FIG. 7D-7F), establishing that CD11b⁺Gr1⁺ cells are indeed functional MDSCs. Using a well-characterized anti-Gr1 neutralizing antibody [18], MDSCs were depleted in mice with established iKRAS PDAC tumors (FIG. 7G). The potent MDSC depletion activity of the anti-Gr1 monoclonal antibody was evidenced by decreased stromal S100A9 expression. This MDSC depletion was accompanied by increased intra-tumoral CD8⁺ T cells, consistent with elimination of the T-cell suppressive activity of MDSCs. It was found that the Gr1-treated mice displayed decreased PDAC progression and resulted in increased OS of PDAC bearing mice, although all of the mice eventually succumbed (FIGS. 8A and 8B). These findings are consistent with prior studies demonstrating that targeted depletion of granulocytic MDSCs can unmask an endogenous T cell response in PDAC, which results in tumor cell death [20].

To evaluate the intrinsic myeloid cell heterogeneity in iKRAS PDAC tumors, clustering was applied based on SNN and 5 myeloid cell clusters were identified with differential expression of signature genes and known functional markers, including Ly6g⁺Cxcr2^{high} (S100A8/A9 expression), Ccl3^{high}Cxcr2^{low} (Mif expression), eosinophils (Siglecf expression), Arg1⁺ and Ccr2⁺ M2 macrophages (FIGS. 8C and 8D, FIGS. 7H and 7I). Given the critical role of CCL2/CCR2 signaling axis in recruitment of myeloid cells to tumors [21] and its high expression on myeloid cells in iKRAS PDAC tumors (FIGS. 7H and 7I), taken together with the finding that depletion of myeloid cells using Gr1 antibody treatment enhanced overall survival of PDAC bearing mice, a CCR2 inhibitor, RS504393, was evaluated in combination with agonist 41BB and antagonist LAG3 antibodies (FIG. 7J). Strikingly, RS504393 in combination with dual ICT (agonist 41BB and antagonist LAG3 antibodies) produced complete regression of established PDAC tumors in all mice. The response was durable with increased OS and 6/10 mice still alive 6 months after initiation of treatment without evidence of relapse (FIGS. 8E and 8F). Treatment with the combination was well tolerated with no treatment-related deaths during the 4 week treatment period.

Pancreatic ductal adenocarcinoma (PDAC) is considered ‘non-immunogenic’ with multiple trials showing its recalcitrance to currently available immune checkpoint therapies including anti-PD1 and anti-CTLA4. Using mouse models of PDAC, surgically resected human PDAC biospecimens and molecular immunology and molecular biology techniques, the inventors identified a novel immunotherapy combination regimen (agonist 41BB mAb+antagonist LAG3 mAb+CCR2 inhibitor) with remarkable and unexpected efficacy at shrinking large established PDAC tumors with durable response in both orthotopic and autochthonous models. In addition, the majority of mice with orthotopic tumors were cured of their disease and rejected tumor cells upon re-challenge with the same tumor cells. The inventors identified that a decrease in myeloid cells and increased CD4⁺ and CD8⁺ T cell infiltrate is seen in the tumor microenvironment after treatment with the aforementioned combination. The inventors also identified a parallel influx of myeloid cells of alternate lineage (monocytic vs. granulocytic) which contribute to adaptive resistance to this combination regimen in PDAC. Together, these unanticipated insights reveal a highly effective and novel triple therapy regimen (agonist 41BB mAb+antagonist LAG3 mAb+CCR2 inhibitor) for treatment of PDAC. These studies also revealed a novel and unexpected resistance mechanism to this triple therapy regimen involving immunosuppressive myeloid cells expressing Cxcr2, arginase 1 (Arg1), and/or inducible nitric oxide synthase (iNOS). Such a discovery informs treatment strategies to inhibit this resistance mechanism involving drugs that target myeloid cells, such as CXCR2 inhibitors, iNOS inhibitors, and/or Arg1 inhibitors.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
(1) Siegel R L, Miller K D, Jemal A. (2020) Cancer statistics, 2020. CA Cancer J Clin. 70(1),7-30
(2) Ying H, Dey P, Yao W, Kimmelman A C, Draetta G F, Maitra A, et al. Genetics and biology of pancreatic ductal adenocarcinoma. Genes Dev 2016; 30(4):355-85.
(3) Kleef J, Korc M, Apte M, La Vecchia C, Johnson C D, Biankin A V, et al. Pancreatic Cancer. Nature Reviews Disease Primers 2016; 2: 16022.
(4) Johnson B A, Yarchoan M, Lee V, Laheru D A, Jaffee E M. Strategies for increasing pancreatic tumor immunogenicity. Clin Cancer Res 2017; 23(7): 1656-1669.
(5) Brahmer J R, Tykodi S S, Chow L Q, Hwu W J, Topalian S L, Hwu P, et al. Safety and activity of anti-PD-L1 antibody in patients with advanced cancer. N Engl J Med 2012; 366(26): 2455-2465.
(6) Royal R E, Levy C, Turner K, Mathur A, Hughes M, Kammula U S, et al. Phase 2 trial of single agent Ipilimumab (anti-CTLA-4) for locally advanced or metastatic pancreatic adenocarcinoma. J Immunotherapy 2010; 33(8): 828-833.
(7) O'Reilly E M, Oh D Y, Dhani N, et al. (2019) Durvalumab With or Without Tremelimumab for Patients With Metastatic Pancreatic Ductal Adenocarcinoma: A Phase 2 Randomized Clinical Trial. JAMA Oncol. 5(10), 1431-1438.
(8) Binnewies M, Roberts E W, Kersten K, Chan V, Fearon D F, Merad M, et al. Understanding the tumor immune microenvironment (TIME) for effective therapy. Nat Med 2018; 24(5): 541-550.
(9) Ying H, Kimmelman A C, Lyssiotis C A, Hua S, Chu G C, Fletcher-Sananikone E, et al. Oncogenic Kras maintains pancreatic tumors through regulation of anabolic glucose metabolism. Cell 2012; 149(3): 656-670.
(10) Bjornson Z B, Nolan G P, Fantl W J. Single-cell mass cytometry for analysis of immune system functional states. Curr Opin Immunol 2013; 25: 484-94.
(11) Carstens J L, Correa de Sampaio P, Yang D, Barua S, Wang H, Rao A, et al. Spatial computation of intratumoral T cells correlates with survival of patients with pancreatic cancer. Nat Commun. 2017; 8: 15095.
(12) Balachandran V P, Luksza M, Zhao J N, et al. (2017) Identification of unique neoantigen qualities in long-term survivors of pancreatic cancer. Nature. 551(7681), 512-6
(13) Trapnell C, Cacchiarelli D, Grimsby J, Pokharel P, Li S, Morse M, et al. The dynamics and regulators of cell fate decisions are revealed by pseudotemporal ordering of single cells. Nature Biotechnology 2014; 32: 381-386.
(14) Eriksson E, Wenthe J, Irenaeus S, Loskog A, Ullenhag S. Gemcitabine reduces MDSCs, Tregs and TGFβ-1 while restoring the Teff/Treg ratio in patients with pancreatic cancer. J Transl Med 2016; 14: 282.
(15) Galluzzi L, Buque A, Kepp O, Zitvogel L and Kroemer G. Immunological effects of conventional chemotherapy and targeted anti-cancer agents. Cancer Cell 2015; 28(6): 690-714.
(16) Suzuki E, Kapoor V, Jassar A S, Kaiser L R, Albelda S M. Gemcitabine selectively eliminates splenic Gr-1+/CD11b+ myeloid derived suppressor cells in tumor-bearing animals and enhances antitumor immunity. Clin Cancer Res 2005; 11(18): 6713-6721.
(17) Newman A, Liu C L, Green M R, Gentles A J, Feng W, Xu Y, et al. Robust enumeration of cell subsets from tissue expression profiles. Nature Methods 2015; 12(5): 453-457.
(18) Wang G, Lu X, Dey P, Deng P, Wu C C, Jiang S, et al. Targeting YAP-dependent MDSC infiltration impairs tumor progression. Cancer Discovery 2016; 6(1): 80-95.
(19) Porembka M R, Mitchem J B, Belt B A, Hsieh C S, Lee H M, Herndon J, et al. Pancreatic adenocarcinoma induces bone marrow mobilization of myeloid-derived suppressor cells which promote primary tumor growth. Cancer Immunol Immunothera 2012; 61(9): 1373-1385.
(20) Stromnes I M, Brockenbrough J S, Izeradjene K, Carlson M A, Cuevas C, Simmons R M, et al. Targeted depletion of an MDSC subset unmasks pancreatic ductal adenocarcinoma to adaptive immunity. Gut 2014; 63(11): 1769-1781.
(21) Gabrilovich D I, Ostrand-Rosenberg S, Bronte V, et al. Coordinated regulation of myeloid cells by tumours. Nat Rev Immunol. 2012; 12(4): 253-68.
(22) Qian B Z, Li J, Zhang H, et al. CCL2 recruits inflammatory monocytes to facilitate breast-tumour metastasis. Nature. 2011; 475(7355):222-225.

What is claimed is:

1. A method for treating a subject for pancreatic ductal adenocarcinoma (PDAC), the method comprising administering to the subject:
(a) a 41BB agonist;
(b) a LAG3 antagonist; and
(c) a chemokine receptor inhibitor.

2. The method of claim 1, wherein the chemokine receptor inhibitor is a CCR1 inhibitor.

3. The method of claim 1, wherein the chemokine receptor inhibitor is a CCR2 inhibitor.

4. The method of claim 3, wherein the chemokine receptor inhibitor is RS504393.

5. The method of any of claims 1-4, further comprising administering to the subject an additional chemokine receptor inhibitor.

6. The method of any of claims 1-5, further comprising administering to the subject an arginase inhibitor.

7. The method of claim 6, wherein the arginase inhibitor is an Arg1 inhibitor.

8. The method of any of claims 1-5, further comprising administering to the subject an iNOS inhibitor.

9. The method of any of claims 1-8, further comprising administering to the subject a Cxcr2 inhibitor.

10. The method of claim 9, wherein the Cxcr2 inhibitor is an anti-Cxcr2 antibody.

11. The method of claim 10, wherein the anti-Cxcr2 antibody is MAB2164.

12. The method of any of claims 6-11, wherein the method comprises inhibiting growth, proliferation, and/or immunosuppressive activity of myeloid cells in the subject.

13. The method of any of claims 1-12, further comprising administering to the subject an additional cancer therapy.

14. The method of claim 13, wherein the additional cancer therapy comprises chemotherapy, radiotherapy, or immunotherapy.

15. The method of claim 14, wherein the additional cancer therapy is chemotherapy.

16. The method of any of claims 13-15, wherein the additional cancer therapy is FOLFIRINOX.

17. The method of any of claims 13-15, wherein the additional cancer therapy is gemcitabine.

18. The method of any of claims 13-15, wherein the additional cancer therapy is gemcitabine with nab-paclitaxel.

19. The method of claim 17 or 18, wherein the additional cancer therapy is administered to the subject prior to administering the 41BB agonist, the LAG3 antagonist, and the chemokine receptor inhibitor.

20. The method of claim 17 or 18, wherein the additional cancer therapy is administered to the subject after administering the 41BB agonist, the LAG3 antagonist, and the chemokine receptor inhibitor.

21. The method of any of claims 1-7, wherein the method does not comprise administering to the subject any additional cancer therapy.

22. The method of any of claims 1-21, wherein the subject was previously treated for PDAC with a previous treatment.

23. The method of claim 22, wherein the subject was determined to be resistant to the previous treatment.

24. The method of claim 22 or 23, wherein the previous treatment comprised FOLFIRINOX.

25. The method of claim 22 or 23, wherein the previous treatment comprised gemcitabine.

26. The method of claim 22 or 23, wherein the previous treatment comprised gemcitabine with nab-paclitaxel.

27. The method of claim 22 or 23, wherein the previous treatment comprised a PD-1 antagonist, a PD-L1 antagonist, or a CTLA-4 antagonist.

28. The method of any of claims 1-27, wherein the 41BB agonist is an anti-41BB antibody.

29. The method of claim 28, wherein the anti-41BB antibody is LOB12.3.

30. The method of any of claims 1-29, wherein the LAG3 antagonist is an anti-LAG3 antibody.

31. The method of claim 30, wherein the anti-LAG3 antibody is C9B7W.

32. The method of any of claims 1-31, wherein the 41BB agonist, the LAG3 antagonist, and the chemokine receptor inhibitor are administered substantially simultaneously.

33. The method of any of claims 1-31, wherein the 41BB agonist, the LAG3 antagonist, and the chemokine receptor inhibitor are administered sequentially.

34. The method of any of claims 1-33, wherein the 41BB agonist, the LAG3 antagonist, and the chemokine receptor inhibitor are administered in a single composition.

35. The method of any of claims 1-33, wherein the 41BB agonist, the LAG3 antagonist, and the chemokine receptor inhibitor are administered in two or more different compositions.

36. A composition comprising:
(a) an anti-41BB agonist;
(b) an anti-LAG3 antagonist; and
(c) a chemokine receptor inhibitor.

37. The composition of claim 36, wherein the 41BB agonist is an anti-41BB antibody.

38. The composition of claim 37, wherein the anti-41BB antibody is LOB12.3.

39. The composition of any of claims 36-38, wherein the LAG3 antagonist is an anti-LAG3 antibody.

40. The composition of claim 39, wherein the anti-LAG3 antibody is C9B7W.

41. The composition of any of claims 36-40, wherein the chemokine receptor inhibitor is a CCR1 inhibitor.

42. The composition of any of claims 36-40, wherein the chemokine receptor inhibitor is a CCR2 inhibitor.

43. The composition of claim 42, wherein the chemokine receptor inhibitor is RS504393.

44. The composition of any of claims 36-43, further comprising an arginase inhibitor.

45. The composition of claim 44, wherein the arginase inhibitor is an Arg1 inhibitor.

46. The composition of any of claims 36-45, further comprising an iNOS inhibitor.

47. The composition of any of claims 36-46, further comprising a Cxcr2 inhibitor.

48. The composition of claim 47, wherein the Cxcr2 inhibitor is an anti-Cxcr2 antibody.

49. The composition of claim 48, wherein the anti-Cxcr2 antibody is MAB2164.

50. The composition of any of claims 36-49, further comprising a pharmaceutically acceptable excipient.

51. A method for treating a subject for pancreatic ductal adenocarcinoma, the method comprising administering to the subject a therapeutically effective amount of:
(a) a 41BB agonist;
(b) a LAG3 antagonist; and
(c) a CCR2 inhibitor.

52. The method of claim 51, wherein the 41BB agonist is an anti-41BB antibody.

53. The method of claim 51, wherein the LAG3 antagonist is an anti-LAG3 antibody.

54. The method of claim 51, wherein the CCR2 inhibitor is RS504393.

55. The method of claim 51, wherein the 41BB agonist is an anti-41BB antibody, the LAG3 antagonist is an anti-LAG3 antibody, and the CCR2 inhibitor is RS504393.

56. A pharmaceutical composition comprising:
(a) a 41BB agonist;
(b) a LAG3 antagonist;
(c) a CCR2 inhibitor; and
(d) a pharmaceutically acceptable excipient.

57. The pharmaceutical composition of claim 56, wherein the 41BB agonist is an anti-41BB antibody.

58. The pharmaceutical composition of claim 56, wherein the LAG3 antagonist is an anti-LAG3 antibody.

59. The pharmaceutical composition of claim 56, wherein the CCR2 inhibitor is RS504393.

60. The pharmaceutical composition of claim 56, wherein the 41BB agonist is an anti-41BB antibody, the LAG3 antagonist is an anti-LAG3 antibody, and the CCR2 inhibitor is RS504393.
